(19) United States
(12) Patent Application Publication
(10) Pub. No.: US 2002/0046418 A1
(43) Pub. Date: Apr. 18, 2002

(54) Controlled environment agreculture bioreactor for heterologous protein production

(76) Inventors: Brian S. Hooker, Kennewick, WA (US); Daniel B. Anderson, Pasco, WA (US); Johnway Gao, Richland, WA (US); Ziyu Dai, Richland, WA (US)

Correspondence Address: Richard C. Peet, FOLEY & LARDNER, Washington Harbour, 3000 K Street, N.W., Suite 500, Washington, DC, 20007-5109, US

(21) Appl. No.: 09/910,958

(22) Filed: Jul. 24, 2001

Related U.S. Application Data
(60) Provisional application No. 60/220,224, filed on Jul. 24, 2000.

(51) Int. Cl. C12N 15/82; A01H 5/00; C12N 15/12; C12N 15/31

(52) U.S. Cl. 800/288; 800/306; 800/317; 800/317.2

(57) ABSTRACT

An integrated system for commercial production of a heterologous protein in transgenic plants under conditions of controlled environment agriculture (CEA) is provided. CEA comprises growth of plants under defined environmental conditions, preferably in a greenhouse, to optimize growth of the transgenic plant as well as expression of the gene encoding the heterologous protein. The transgenic plants used in the present invention are transformed with an expression vector comprising a CEA promoter operably linked to a gene encoding the heterologous protein of interest, wherein the CEA promoter is selected to maximize heterologous protein production under the defined environmental conditions of CEA.

This is application claims priority to U.S. application Ser. No. 60/220,224 filed Jul. 24, 2000 that is herein incorporated by reference.

### FIELD OF THE INVENTION

The present invention relates generally to an integrated system for commercial production of a heterologous protein in transgenic plants under conditions of controlled environment agriculture (CEA). CEA comprises growth of plants under defined environmental conditions, preferably in a greenhouse, to optimize growth of the transgenic plant as well as expression of the gene encoding the heterologous protein. The transgenic plants used in the present invention are transformed with an expression vector comprising a CEA promoter operably linked to a gene encoding the heterologous protein of interest, wherein the CEA promoter is selected to maximize heterologous protein production under the defined environmental conditions of CEA.

In CEA, the transgenic plants may be cultivated through hydroponics or in soil-less or soil-containing media. The transgenic plants selected for heterologous protein production under the defined environmental conditions of CEA may also be grown in open field agriculture (OFA) to produce the protein of interest. Diverse plant species may be used including dicots and monocots.

The protein production system of the present invention comprises a transgenic plant transformed with an expression vector comprising a CEA promoter operably linked to a gene encoding the heterologous protein of interest. Preferably, the plant used in this protein production system is selected because under conditions of CEA it produces (1) rapid and efficient growth of harvested plant biomass containing the heterologous protein; (2) large amounts of heterologous protein in the harvested plant biomass; and (3) plant tissue or plant tissue extract wherein the heterologous protein is stable. Also desirable is a CEA plant that is efficiently transformed, selected and propagated so that plants used in the heterologous protein production system can be rapidly grown to facilitate continuous production of recombinant protein product.

### BACKGROUND OF THE INVENTION

Many diverse methods and hosts have been tested for the commercial production of heterologous proteins in transgenic organisms. These diverse methods and hosts include transgenic single cell systems such as bacteria, fungi, animal and plant cells, as well as transgenic whole organism systems such as transgenic plants, insects and animals.

Fermentation techniques for large-scale production of proteins in bacteria, fungi and higher organism cell cultures are well established. The capital costs associated with establishment and maintenance of fermentation facilities, however, are substantial. Similarly, the production of various heterologous proteins in transgenic animals has been described but the cost of this approach is prohibitive for all but very high value proteins.

The use of a transgenic plant as a bioreactor for production of a heterologous protein has received considerable attention. Heterologous proteins have been expressed in whole plants and selected plant organs. In principal, plants represent a highly effective and economical means to produce recombinant proteins because they can be grown on a large scale with modest cost inputs. Most commercially important plant species can now be transformed. In addition, for pharmaceutical applications, the heterologous proteins produced in plants are free from human pathogen contamination.

A number of different strategies have been used to produce heterologous proteins and peptides in plants. A gene of interest may be operably linked to a constitutive promoter such that a plant transformed with this DNA construct produces the heterologous protein encoded by the gene continuously, in all portions of the plant. Alternatively, the gene of interest may be operably linked to a tissue-preferred promoter such that a plant transformed with this DNA construct produces the heterologous protein encoded by the gene in a specific tissue. See, for example, U.S. Pat. No. 5,767,379. Another approach to heterologous protein production is to fuse a structural gene encoding the heterologous protein in frame with a second gene so that a plant transformed with this DNA construct expresses a fusion protein. The fusion protein can be isolated and processed to produce the heterologous protein of interest. See, for example, U.S. Pat. No. 5,977,438. Genes encoding heterologous proteins that have been successfully expressed in plant cells include those from bacteria, animals, fungi and other plant species.

There are now many examples of successful use of plants or cultured plant cells to produce active mammalian proteins, enzymes, vaccines, antibodies, peptides, and other bioactive species. Ma et al., Science 268: 716-719 (1995), first described the production of a functional secretory immunoglobulin in transgenic tobacco. Genes encoding the heavy and light chains of a murine antibody, a murine joining chain, and a rabbit secretory component were introduced into separate transgenic plants. Through cross-pollination, plants were obtained that co-express and correctly assemble all components and produce a functionally active secretory antibody. In another study, a method for producing antiviral vaccines by expressing a viral protein in transgenic plants was described. Mason et al., Proc. Natl. Acad. Sci. U.S.A. 93: 5335-5340 (1996).

Alternatively, the production and purification of a vaccine may be facilitated by engineering a plant virus that carries a mammalian pathogen epitope. By using a plant virus, the accidental shedding of a virulent virus that is a human pathogen with the vaccine is avoided, and the same plant virus may be used to vaccinate several hosts. See, for example, U.S. Pat. No. 5,889,190.

In a study aimed at improving the nutritional status of pasture legumes, a sulfur-rich seed albumin from sunflower was expressed in the leaves of transgenic subterranean clover. Khan et al., Transgenic Res. 5:178-185 (1996). By targeting the recombinant protein to the endoplasmic reticulum of the transgenic plant leaf cells, an accumulation of transgenic sunflower seed albumin up to 1.3% of the total extractable protein was achieved.

OFA has been proposed for the commercial production of heterologous proteins in transgenic plants because of its relatively low cost. Following seed increase, a transgenic plant expressing the heterologous protein of interest can be grown on many acres in OFA to produce plant biomass from which the heterologous protein is purified. OFA for heterologous protein production, however, has many disadvantages. OFA is frequently unreliable because changes in growing conditions can dramatically affect yield of plant biomass and/or heterologous protein. Furthermore, seasonal weather changes make it difficult or impossible to continuously cultivate transgenic plants for heterologous protein production. This requires large and costly infrastructure to extract and purify targeted proteins from large, infrequent harvests. Additionally, some pharmaceuticals must be produced under stringently controlled environmental conditions wherein the effect of adventitious agents can be minimized. These stringently controlled environmental conditions can be created in a CEA production system where frequent harvest of relatively small crops will aid in reducing size and cost of equipment required for downstream processing.

Another disadvantage of OFA for heterologous protein production is that it is more difficult to prevent the gene encoding the protein of interest from being introduced into related or wild species through cross pollination. Likewise, there is an increased risk that transgenic plants grown in OFA could enter the food or feed chain. These are issues of concern to government regulatory agencies and the general public. OFA systems are also more susceptible to sabotage and bioterrorism attacks.

There is a need, therefore, for transgenic plant systems that overcome the above limitations. There is a need for a transgenic plant system that produces a heterologous protein of interest consistently, safely and reliably, with high yields, and at low cost.

### SUMMARY OF THE INVENTION

It is an object of the present invention to provide a method for developing a transgenic plant system, consisting of plants genetically transformed for foreign protein expression grown in a controlled environment, for reliable and continuous production of a heterologous protein. It is another object of the present invention to provide a method for selecting a transgenic plant that optimally produces heterologous protein in a continuous CEA production system.

These and other objects are achieved, in one aspect of the present invention, by providing a plant system for producing a heterologous protein under defined environmental conditions of CEA, the plant system comprising a plant (a) transformed with an expression vector comprising a gene coding for the heterologous protein operably linked to a promoter that is selected for optimal expression under the defined environmental conditions; (b) that produces a large amount of plant biomass under the defined environmental conditions of CEA, and (c) that produces a plant tissue or tissue extract wherein the heterologous protein is stable. The defined environmental conditions under which the transgenic plant is grown are optimized to achieve maximum yield of the plant tissue in which the heterologous protein is preferentially expressed. Also provided is a plant system wherein the plant is selected from the group consisting of Solanum, Spinacia and Brassica. The plant system may be Solanum; a light-inducible promoter such as the promoter from the Rubisco promoter, and the defined environmental conditions of CEA include at least 12 hours of light per day.

Also provided is a plant system wherein the promoter is CO₂-inducible and the defined environmental conditions of CEA include between 350 and 2,500 ppm CO₂. The plant system may also include a heat-inducible promoter and the defined environmental conditions of CEA include a temperature between 25 and 40° C., optimally between 37 and 40° C. The plant system may include a heat-inducible promoter from the hsp80 gene.

Another aspect of the present invention is a method of producing heterologous protein in a transformed plant comprising the steps of (a) transforming a plant with an expression vector comprising a gene coding for the heterologous protein operably linked to a promoter that is selected for optimal expression under defined environmental conditions of CEA; (b) cultivating the plant under the defined environment conditions; and (c) extracting the heterologous protein. The plant may be selected from the group consisting of Solanum, Spinacia and Brassica. Furthermore, the plant may be Solanum, the promoter is light-inducible and the defined environmental conditions of CEA include at least 12 hours of light per day. The promoter may be from the Rubisco small subunit gene.

Another aspect of the invention involves use of a CO₂-inducible promoter and the defined environmental conditions of CEA include between 350 and 2,500 ppm CO₂, preferably between 500 and 2,000 ppm, more preferably between 1,000 and 1,500 ppm. Furthermore, the promoter may be heat-inducible and the defined environmental conditions of CEA include a temperature between 25 and 40° C., more perferably between 30 and 40° C., optimally between 37 and 40° C. The heat-inducible promoter may be the promoter from the hsp80 gene.

Another aspect of the invention provides a method of making a plant system for production of a heterologous protein comprising the steps of (a) identifying a plant that produces a large amount of plant biomass under defined environmental conditions of CEA; (b) transforming the plant with an expression vector comprising a gene coding for the heterologous protein operably linked to a promoter that is selected for optimal expression under the defined environmental conditions of CEA; and (c) selecting a transformed plant that (i) produces a large amount of the heterologous protein and (ii) the heterologous protein is stable in the tissue or an extract made from the plant. The plant may be selected to produce a plant biomass of between about 0.2 and 5 kg fresh weight vines per plant for potato or between about 0.2 and 250 grams dry weight per plant for mustard. The plant may be selected to produce between about 10 and 1300 kg heterologous protein/acre/year for potato, or between about 8 and 1000 kg/acre/year heterologous protein for mustard. The method may involve the plant Solanum, a light-inducible promoter and the defined environmental conditions of CEA include at least 12 hours of light per day The method may involve the promoter from the ribulose bis-phosphate carboxylase (Rubisco) small subunit gene. The method may involve a CO₂-inducible promoter and the defined environmental conditions of CEA include between 350 and 2,500 ppm CO₂. The method may involve the heat-inducible promoter and the defined environmental conditions of CEA include a temperature between 25 and 40° C., optimally between 37 and 40° C. The heat-inducible promoter may be promoter from the hsp80 gene.

### BRIEF DESCRIPTION OF THE DRAWINGS

FIG. 1. Plasmid map of pZD424 comprising the RbcS-3C promoter operably linked to GUS coding sequence and the nos promoter operably linked to nptII selectable marker.

FIG. 2. Plasmid map of pZD424L34 comprising the nptII selectable marker operably linked to tobacco rpL34 promoter.

FIG. 3. Propagation of potato shoots arising from A. tumefaciens—transformed potato stem internode explants on solid medium in magenta box.

FIG. 4. Constructs used for Agrobacterium-mediated transformation. Cassettes contain left border sequence (LB), nopaline synthase promoter, neomycin phosphotransferase II gene (NPTII), nopaline synthase terminator, Rubisco small subunit promoter (RbcS-3C), 5′-untranslated leaders (AMV, RbcS-3C leaders), transit peptides (sporamin A or RbcS-2A), E1 coding sequence, transcription terminators (T7-T5), and right border sequence (RB). ra-chl, and rr-vac are listed as designations for the two different transgene expression constructs.

FIG. 5. E1 activity of different individual transgenic plants bearing different expression cassettes. Panel (A) and (B): E1 coding sequence under the control of leaf specific RbcS-3C promoter, and its 5′-untranslated leader with the signal peptide sequence of a sporamin (rr-vac) or AMV 5′-untranslated leader with a chloroplast signal peptide (ra-chl).

FIG. 6. The expression of the E1 gene in selected transgenic potato plants possessing higher E1 activity. (A) RNA gel-blot of wild type and E1 expressing selected transgenic potato plants. RNA gel-blot contains 20 μg per lane probed with a 1.2 kb Xba I/BamH I E1 coding sequence fragment labeled with [α-³²P]-dCTP. The RNA isolated from leaf tissues of wild-type potato plant served as the control. Lanes representing individual transgenic plants are indicated by transformant designation and transgenic plant number. F precede the transgenic plant identifier correspond to potato FL1607. (B) immunoblot detection of E1 protein expressed in leaf tissues of selected transgenic plants. Forty micrograms of total leaf soluble protein extract from wild-type potato or selected transgenic potato plants were analyzed by immunoblotting with monoclonal antibodies against full-length E1 protein. Fifty, one hundred, and two hundred micrograms of E1 protein were used for positive controls and served as a standard series for estimation of E1 protein in leaf protein extract, which was purified from culture supernatant of streptomyces lividans carrying a plasmid containing a 3.7 kb genomic fragment of A. cellulolyticus E1 gene. The negative control was the protein extract from wild-type potato plants. Lanes correspond to individual transgenic plants as indicated by the transformant designation and transgenic plant number

FIG. 7. Average cellulase activity for two tested plant lines resulting from two-week incubation under 24- and 12-hour photoperiods.

FIG. 8. Average cellulase yield per plant for the two tested plant lines resulting after four-week incubation under 24- and 12-hour photoperiods.

FIG. 9. (A) mustard primary transformed shoots on stage I medium; (B) mustard primary transformed shoots excised from green callus originating on transformed explants also on stage I medium; and (C) mustard primary transformed shoots in rooting medium.

FIG. 10. Factor VIII proteolytic stability studies in extracts of FL1607 Potato and alfalfa. Error bars correspond to standard deviation from reported average values from three separate experiments.

FIG. 11. Western blot immunoassays completed on FL1607 potato (Solanum tuberosum L. cv. FL1607) extracts resulting from above-described proteolytic stability tests. Lane 1 in each blot corresponds to the factor VIII standard and subsequent even lanes (2, 4, 6, etc.) correspond to factor VIII in descending order (odd numbered only) leaf extract at 0 hours incubation; subsequent odd lanes (3, 5, 7, etc.) correspond to factor VIII in descending order (odd numbered only) leaf extract at 2 hours incubation.

FIG. 12. Western blot immunoassays completed on alfalfa (Medicago sativa L.) extracts resulting from above-described proteolytic stability tests. Lane 1 in each blot corresponds to factor VIII standard; subsequent even lanes (2, 4, 6, etc.) correspond to factor VIII in descending order leaf extract at 0 hours incubation; subsequent odd lanes (3, 5, 7, etc.) correspond to factor VIII in descending order leaf extract at 2 hours incubation.

### DETAILED DESCRIPTION OF THE PREFERRED EMBODIMENTS

The present invention provides an integrated system for commercial production of a heterologous protein in transgenic plants. The present invention, utilizing the defined environmental conditions of CEA, provides a productivity of up to 1300 kg/acre/year recombinant protein in potato foliage and 1000 kg/acre/year in brassica foliage. This is over two orders of magnitude higher than recombinant protein productivities previously reported for OFA, including 5 kg/acre/year for corn (Mison et al., Biopharm, 13:48-54, 2000), 30 kg/acre/year for tobacco (Calculated from tobacco phytase expression levels [Verwoerd et al., Plant Physiol., 109_{—}1199-1205, 1995] and biomass yield [Oishi, presentation at Ag Biotech World Forum, Las Vegas, Nev., February, 2000]) and 27 kg/acre/year for alfalfa (Austin-Phillips et al., U.S. Pat. No. 6,248,938, 2001). This dramatic increase in productivity allows for the production of recombinant protein in CEA at a cost that is competitive with that associated with OFA with the additional benefits associated with CEA including barriers against pest and disease infestation, precise control over process inputs and outputs for regulatory approval purposes, prevention of issues of “genetic drift” into and from other plant species and protection against unpredictable weather conditions, among others. The present invention provides for novel methods for the selection of suitable plant species or cultivars for production of heterologous proteins; expression vectors comprising a CEA promoters operably linked to genes coding for heterologous proteins of interest, the use of defined environmental conditions for CEA, and a continuous heterologous protein production process.

Preferably, a plant species or cultivar is selected for use in the integrated system because it is efficiently transformed with an expression vector comprising the gene coding for the heterologous protein. Efficient transformation with an expression vector carrying a gene encoding the heterologous protein provides for rapid production of numerous plants that can be screened for high expression of heterologous protein as well as other characteristics useful for the commercial production of the protein of interest. Preferably, the selected transformed plants produce plant tissues and a plant extract in which the heterologous protein is stable.

The CEA promoter is selected to optimize expression of the gene coding for the heterologous protein of interest under the defined environmental conditions of CEA. For example, to increase plant growth rate, a transgenic plant may be cultivated for extended photoperiods. Under these light conditions, a light-inducible promoter, such as the ribulose bis-phosphate carboxylase (RuBisco) small subunit promoter, can be selected as the CEA promoter to optimize expression of the gene coding for the heterologous protein.

The plant system of the instant invention circumvents the limitations imposed by natural crop growth cycles. By producing the transgenic plant under defined environmental conditions of CEA in a greenhouse, the transgenic plant can be cultivated at any time of the year under conditions that optimize production of plant biomass. As a consequence, the integrated system of the instant invention provides a continuous supply of the heterologous protein without the seasonal disruptions associated with an OFA system. Once the transgenic plant containing the heterologous protein of interest is harvested, these plants are immediately replaced with new transgenic plants so that the integrated system can be used on a continuous basis. This system allows for efficient and continuous processing of plant biomass thereby increasing the annual protein productivity rate and minimizing equipment size and capital costs associated with downstream processing

1. Selection of Plants for the Integrated System

A suitable plant is selected for fast and efficient propagation and growth under the defined environmental conditions of CEA. Generally, vegetative propagation of the selected plant is preferred unless the selected plant is a hybrid or is genetically homozygous and can be reproduced by selfing. Vegetative propagation methods are selected and developed to minimize somatic variability in “progeny” (i.e., techniques that avoid formation of undifferentiated tissues such as callus).

Under the CEA conditions, the plant produces large amounts of plant tissue that is rich in heterologous protein. In general, the growth characteristics of the plant to be used in the invention are known to the skilled person. These growth conditions will serve as the basis for selecting a suitable plant as well as the growth conditions for CEA.

A suitable plant for the invention will also have desirable transformation characteristics. For example, high transformation efficiency with the vector is preferred. Efficient transformation permits rapid screening of large numbers of presumptively transformed lines for desired characteristics including efficient CEA promoter expression under defined environmental conditions of CEA, production of large amounts of plant biomass, production of large amounts of heterologous protein in the plant biomass and stability of the heterologous protein in plant tissues and extracts made from the harvested plant biomass. As a result of the above selection process, the plant according to the present invention, when cultivated under the preferred CEA conditions, produces large amounts of appropriate plant tissue, and therefore large amounts of the heterologous protein or peptide of interest.

A plant suitable for use in the integrated system of the present invention can be a monocot or dicot plant. A suitable plant for use in the present invention may be an annual or a perennial plant. Preferably, transgenic plants used in the present invention are grown under defined environmental conditions such as in a greenhouse. The plants may be cultivated hydroponically or in solid medium that can include soil-less or soil-containing media. When sufficient plant biomass has been obtained, the transgenic plants, or relevant plant tissues from the transgenic plants, are harvested for extraction of the heterologous protein. The harvested plants can be immediately replaced in the greenhouse, thereby providing an integrated system for continuous cultivation of transgenic plants.

According to a preferred embodiment, a plant suitable for the present invention is a Solanaceae plant, a Brassicaceae plant, or a Chenopodiace plant. More preferably, a plant suitable for the present invention is a Solanum plant, a Brassica plant, or a Spinacia plant. Particularly preferred, the plant may be a S. tuberosum plant, a B. juncea plant, a B. chinensis plant, a B. rapa plant, a B. oleracea plant, or a S. oleracea plant. Still more preferably, the plant may be a S. tuberosum L.cv, FL1607 plant, a B. juncea L.cv. Czerniak plant, a B. oleracea L.cv. viridis plant., a B. chinensis plant, and a B. rapa plant.

According to another preferred embodiment of the invention, the plant biomass produced in the expression system is between 0.2 and 5; preferably about 0.5, more preferably about 1.0, optimally more than 1.0 kg fresh weight vines per plant for potato. According to another preferred embodiment of the invention, the plant biomass produced in the expression system is between 0.2 and 250; preferably about 10; more preferably about 30; optimally greater than 62 grams dry weight mustard greens per plant.

Particularly preferred are plants that can be grown efficiently in the presence of extended photoperiods. These plants are transformed with an expression vector comprising a light-inducible promoter operably linked to a gene coding for a heterologous protein. S. tuberosum plants may be grown in the light for at least 12 hours per day, at least 14 hours per day; at least 16 hours per day; preferably at least 18 hours per day; more preferably at least 20 hours per day; most preferably 22 hours per day; and optimally at least 24 hours per day. The S. tuberosum plant is grown between 20 and 30° C., preferably between 22 and 28° C.; more preferably between 24 and 26° C. and most preferably at 24° C.

Spinacia oleracea plants may be grown in the light for at least 8 hours per day, preferably at least 10 hours per day; more preferably at least 12 hours per day; most preferably at least 14 hours per day; optimally at least 16 hours per day. The Spinacia plant is grown between 20 and 30° C., preferably between 22 and 28° C.; more preferably between 24 and 26° C. and most preferably at 24° C.

B. juncea plants may be grown in the light optimally at about 9 to 10 hours per day, preferably for at least 9 hours per day, at least 11 hours per day; at least 13 hours per day; preferably at least 15 hours per day; preferably at least 17 hours per day; and preferably 19 hours per day. The Brassica plant is grown between 20 and 30° C., preferably between 22 and 28° C.; more preferably between 24 and 26° C. and most preferably at 24° C.

B. oleracea var. acephala; B. oleracea var. alboglabra; B chinensis and B. parachinenesis plants may be grown in the light for at least 8 hours per day, at least 10 hours per day; at least 12 hours per day; preferably at least 14 hours per day; more preferably at least 16 hours per day; most preferably 18 hours per day; and optimally at about 20 hours per day. The Brassica plant is grown between 20 and 30° C., preferably between 22 and 28° C.; more preferably between 24 and 26° C. and most preferably at 24° C.

Another preferred embodiment involves the production of between 10 and 1300; preferably about 50; more preferably about 100; more preferably about 200; more preferably about 300; optimally about 350 or more kilograms per acre per year heterologous protein in transgenic potato. Another preferred embodiment involves the production of between 8 and 1000; preferably about 50; more preferably about 100; more preferably about 200; optimally about 220 or more kilograms per acre per year heterologous protein in transgenic brassica.

2. Production of Transgenic Plants Expressing the Desired Heterologous Protein

The present invention utilizes a transgenic plant for the production of a heterologous protein of interest. The transgenic plant is transformed with an expression vector comprising a promoter operably linked to a gene encoding the heterologous protein. The promoter may be constitutive, tissue-preferred or inducible. Accordingly, the expression of the gene coding for the heterologous protein or peptide of interest can be carefully regulated. Preferably, the promoter is selected for optimal expression under the defined environmental conditions of the CEA. The transgenic plant may be transformed with more than one expression vector, each of which carries a different gene that codes for a unique heterologous protein or peptide. Alternatively, the transgenic plant may be transformed with one expression vector carrying more than one gene coding for a heterologous protein.

a. The Expression Vector

An expression vector according to the instant invention comprises the regulatory sequences necessary for expression of a gene coding for the heterologous protein of interest. Many expression vectors for use in plants are known to the skilled artisan. For example, Gruber et al., “Vectors for Plant Transformation,” in METHODS IN PLANT MOLECULAR BIOLOGY AND BIOTECHNOLOGY, Glick et al. (eds.), pages 89-119 (CRC Press, 1993), provides a general description of plant expression vectors.

An expression vector comprises a DNA sequence coding the heterologous protein of interest operably linked to a promoter and a transcription termination sequence. The expression vector may also comprise a selectable marker or screenable marker. In general, an expression vector comprises a cloning site for the insertion of a gene coding for the heterologous protein. These and other elements that may comprise the expression vector are discussed in detail below. The “heterologous gene” or “heterologous DNA” that codes for a heterologous protein includes any gene that has been isolated and then transformed into the selected host plant and therefore includes genes isolated from the selected host plant.

“Operably linked” refers to components of an expression vector that function as a unit to express a heterologous protein. For example, a promoter operably linked to a heterologous gene that codes for a protein, promotes the production of functional mRNA corresponding to the heterologous gene.

The expression vector may also comprise a selectable or screenable marker gene to facilitate selection and detection of transformed plant cells. In accordance with this invention, a selectable marker gene codes for a protein that confers resistance or tolerance to a toxic chemical such as an antibiotic or herbicide. In accordance with this invention, a screenable marker gene encodes a protein that confers a unique phenotype, such as a different color to transformed cells.

Acceptable selectable marker genes for plant transformation are well known in the art. For example, a general review of suitable markers for the members of the grass family is found in Wilmink and Dons, Plant Mol. Biol., Reptr, 11 (2):165-185(1993). Weising et al., Annual Rev. Genet. 22:421 (1988) describes selectable marker genes useful for transformation of dicot plants. Examples of suitable selectable marker genes are the neo gene described by Beck et al., Gene 19:327 (1982) and Fraley et al., CRC Critical Reviews in Plant Science 4:1 (1986); the hygromycin resistance gene described in Rothstein et al., Gene 53: 153-161 (1987) and Hagio et al., Plant Cell Reports 14:329 (1995); the bar gene described by Thompson et al., EMBO Journal 6: 2519-2523 (1987) and Toki et al., Plant Physiol. 100:1503 (1992), among others. See, generally, Yarranton, Curr. Opin. Biotech. 3:506 (1992); Chistopherson et al., Proc. Natl. Acad. Sci. USA 89:6314 (1992); Yao et al., Cell 71:63 (1992) and Reznikoff, Mol. Microbiol. 6:2419 (1992).

Examples of suitable screenable marker genes are the gus gene described by Jefferson et al., Proc. Natl. Acad. Sci. USA 6:3901 (1986), the luciferase gene taught by Ow et al., Science 234:856 (1986), and the green fluorescent protein gene described by Chalfie et al., Science 263: 802-805 (1994).

The expression vectors may also include sequences that allow their selection and propagation in a secondary host, such as, sequences containing a bacterial origin of replication and a selectable marker gene. Typical secondary hosts include bacteria and yeast. In one embodiment, the secondary host is Escherichia coli, the origin of replication is a colE1-type, and the selectable marker gene codes for ampicillin resistance. Such expression vectors are well known in the art.

The expression vectors of the present invention may be based on the Agrobacterium tumefaciens Ti vector containing a T-DNA border region into which the gene of interest is inserted. The construction of Ti-based vectors is well known in the art and are described in detail in Sheng, J. and Citovsky, V., Plant Cell 8:1699-1710 (1996). Many Agrobacterium strains are known in the art, particularly for dicot plant transformation, and can be used in the methods of the invention. See, for example, Hooykaas, Plant Mol. Biol. 13, 327 (1989); Smith et al, Crop Science 35: 301 (1995); Chilton, Proc. Natl. Acad. Sci. USA 90: 3119 (1993); Mollony et al., Monograph Theor. Appl. Genet NY 19: 148 (1993); Ishida et al., Nature Biotechnol. 14 745 (1996); and Komari et al., The Plant Journal 10: 165 (1996).

The expression vector may also include a DNA sequence that promotes integration of heterologous DNA into the plant genome. DNA sequences that may promote integration of the expression vector into the plant genome include a transposon.

b. The Gene Coding for a Heterologous Protein or Peptide

A skilled artisan recognizes that many heterologous proteins may be produced using the plant system of the present invention. Any gene coding for a heterologous protein of interest may be suitable for expression using the instant invention. A skilled person would recognize that a cDNA of the desired heterologous coding sequence is preferred for the invention. The heterologous coding sequence may be for any protein of interest, cloned from a prokaryotic or eukaryotic host. The gene providing the desired product will particularly be those genes associated with commercial products. Therefore, products of particular interest include, but are not limited to, enzymes, such as chymosin, proteases, polymerases, saccharidases, dehydrogenases, nucleases, glucanase, glucose oxidase, α-amylase, oxidoreductases (such as fungal peroxidases and laccases), xylanases, phytase, cellulase, hemicellulase, and lipase. More specifically, the invention can be used to produce enzymes such as those used in detergents, rennin, horse radish peroxidase, amylases from other plants, soil remediation enzymes, and other such industrial proteins.

Other proteins of interest are mammalian proteins. These proteins particularly may be used as pharmaceuticals. Such proteins include, but are not limited to blood proteins (such as, serum albumin, Factor VII, Factor VIII, Factor IX, Factor X, Factor XIII, fibrinogen, fibronectin, thrombin, tissue plasminogen activator, Protein C, von Willebrand factor, antithrombin III, and erythropoietin), colony stimulating factors (such as, granulocyte colony-stimulating factor (G-CSF), macrophage colony-stimulating factor (M-CSF), and granulocyte macrophage colony-stimulating factor (GM-CSF)), cytokines (such as, interleukins), integrins, addressing, selecting, homing receptors, surface membrane proteins (such as, surface membrane protein receptors), T cell receptor units, immunoglobulins, soluble major histocompatibility-complex antigens, structural proteins (such as, collagen, fibroin, elastin, tubulin, actin, and myosin), growth factor receptors, growth factors, growth hormone, cell cycle proteins, vaccines, cytokines, hyaluronic acid and antibodies.

The present invention may also produce polypeptides useful for veterinary use such as vaccines and growth hormones. The products can then be formulated into a mash product or formulated seed product directly useful in veterinary applications.

The heterologous protein may be modified, using methods well known to those skilled in the art, to reduce or eliminate immunogenic sensitization reactions in humans. For example, the heterologous protein may be a humanized monoclonal antibody against a cancer-specific antigen.

A skilled artisan will also understand that a protein of interest may be produced with different, but functionally equivalent nucleotide molecules. Two nucleotide sequences are considered to be “functionally homologous” if they hybridize with one another under moderately stringent conditions, e.g. 0.1% SSC at room temperature. Typically, two homologous nucleotide sequences are greater than or equal to about 60% identical when optimally aligned using the ALIGN program (Dayhoff, M. O., in ATLAS OF PROTEIN SEQUENCE AND STRUCTURE (1972) Vol. 5, National Biomedical Research Foundation, pp. 101-110, and Supplement 2 to this volume, pp. 1-10.) Likewise, the nucleotide sequence coding for the protein of interest may be synthesized to reflect preferred codon usage in plants. See, for example, Murray et al., Nucleic Acids Res. 17: 477-498 (1989).

C. A Targeting Sequence

In addition to encoding the protein of interest, the expression vector may also code for a targeting sequence that increases protein stability or allows increases protein stability, post-translational processing and/or translocation of the protein, as appropriate. By employing the signal peptide, the protein of interest may be translocated from the cells in which they are expressed or sequestered in a specific subcellular compartment. While it is riot required that the protein be secreted from the cells in which the protein is produced, this often facilitates the isolation and purification of the recombinant protein. For example, an apoplast-specific cleavage transit peptide, such as a pathogenesis related II transit peptide, may be employed to direct the secretion of the heterologous protein into the plant root zone. Those of skill in the art can identify other suitable signal peptides to be used with this invention. See, for example, Jones et al., Tansley Review 17:567-597 (1989).

d. The CEA Promoter

The defined environmental conditions of the CEA can include many hours of continuous light. Under these conditions, a light-inducible CEA promoter is used to maximize expression of the heterologous protein. Light-inducible promoters are well known in the art. A preferred promoter for the present invention is a light-inducible promoter from a gene which is highly expressed in leaf tissue. A ribulose 1,5-diphosphate carboxylase small subunit (Rubisco) promoter is particularly preferred. Another preferred light-inducible promoter is the promoter from the chlorophyll a/b-binding protein that is also highly expressed in leaf tissue. Broglie et al., Biotech. 1: 55 (1988); Manzara et al., Plant Cell 3: 1305 (1991); Kojima et al., Plant Mol. Biol., 19: 405 (1992); Lamppa et al., Mol. Cell. Biol. 5: 1370 (1985) and Sullivan et al., Mol. Gen. Genet. 215: 431 (1989). Other light-inducible promoters that can be used in the present invention include the promoters from the phosphoenolpyruvate carboxylase gene; the PsaD gene; the pea plastocyanin gene and the PSI-D gene. Schaffner et al. Plant J 2: 221-232 (1992);; Flieger et al. Plant J 6: 359-368 (1994); Pwee et al. Plant J 3: 437-449 (1993)and Yamamoto et al. Plant Mol Biol 22: 985-994(1993).The defined environmental conditions of the CEA might include elevated concentrations of carbon dioxide that induce expression of a carbon dioxide-inducible CEA promoter. Carbon dioxide-inducible promoters, for example Rubisco in tomato and various in Sinechococcus sp. (cyanobacteria), are known in the art. Murchie et al., Plant Physiol Biochem 37: 251-260 (1999). Scanlan et al., Gene 90: 43-49 (1990).

Alternatively, the defined environmental conditions of the CEA might include high temperatures. If the transgenic plant is grown at a sufficiently high temperature, the heat-inducible promoter will induce expression of a heat sensitive gene. The heat-inducible promoter might be the promoter from the heat shock 80.5 (hsp80) protein. See, for example, U.S. Pat. No. 5,187,267.

The plant can be treated with chemicals that induce expression of an inducible promoter. For example, the plant can be treated with salicylic acid or methyl jasmonate to induce promoter expression related to the pathogenesis-related beta-1,3-glucanase and lipoxygenase 1 genes, respectively. See, for example, Shah et al., Plant J. 10: 1089 (1996).

e. Other Suitable Promoters

Alternative promoters that are not tied to a particular CEA condition may also be useful in the defined conditions of CEA, given the ability to efficiently produce heterologous protein-bearing plant biomass. In this embodiment, a heterologous gene may be operably linked to a constitutive promoter so that the heterologous protein is produced relatively constantly in all tissues of the plant. A constitutive promoter is a promoter where the rates of RNA polymerase binding and transcription initiation are approximately constant and relatively independent of external stimuli. Examples of constitutive promoters include the cauliflower mosaic virus (CaMV) 35S and 19S promoters described by Poszkowski et al., EMBO J., 3:2719 (1989) (original sequence of CaMV—Gardner et al. Nucleic Acids Res. 9: 2871-2888 (1981); original sequence of CaMV 35S in vector—Sanders et al. Nucleic Acids Res. 15: 1543-1558(1987).) and Odell et al., Nature, 313:810 (1985), the nos promoter from native Ti plasmids of A. tumefaciens described by Herrera-Estrella, et al., Nature 303:209-213 (1983), and the 2′ promoter taught by Velten, et al., EMBO J. 3, 2723-2730 (1984).

A promoter suitable for the instant invention may also be a tissue-preferred promoter. A tissue-preferred promoter has selectively higher activities in certain tissues than in others and controls transcription by modulating RNA polymerase binding at a specific time during development, or in a tissue-specific manner. Many examples of tissue-preferred promoters are known to the skilled person. Some examples are given in Chua et al., Science 244:174-181 (1989).

A hybrid promoter may also be used for the present invention. A hybrid promoter operatively combines a core promoter from one promoter, such as a strong, constitutive promoter of CaMV, with regulatory elements from another promoter, such as a tissue-preferred or inducible promoter. Hybrid promoter allows for more flexible control in both the expression level and expression pattern of the gene under its control. Examples of hybrid promoters are described in U.S. Pat. No. 5,962,769.

f. Transcription and Translation Termination Sequences

The expression cassettes or chimeric genes of the present invention typically have a transcriptional termination region at the opposite end from the transcription initiation regulatory region. The transcriptional termination region may normally be associated with the transcriptional initiation region or from a different gene. The transcriptional termination region may be selected, particularly for stability of the mRNA to enhance expression. Illustrative transcriptional termination regions include the NOS terminator from the Agrobacterium Ti plasmid and the rice alpha-amylase terminator.

Polyadenylation tails are also commonly added to the expression cassette to optimize high levels of transcription and proper transcription termination. Alber and Kawasaki, Mol. and Appl. Genet. 1:419-434 1982. Polyadenylation sequences include, but are not limited to, the Agrobacterium octopine synthetase gene from Gielen et al., EMBO J. 3:835-846 (1984) or the gene of the same species Depicker, et al., Mol. Appl. Genet. 1:561-573 (1982).

9. Plant Transformation

According to the present invention, it is preferred to use a plant that can be transformed with high transformation efficiency. Transformation efficiency varies according to the specific plant species and the transformation technique used. In general, transformation efficiency is defined as the number of transgenic plants that can be obtained per transformed ex-plant.

High transformation efficiency provides for continuous production of transgenic plants using newly transformed and regenerated plants without relying on conventional plant propagation techniques.

Expression vectors containing the gene for a heterologous protein of interest can be introduced into plant cells by a variety of techniques. For example, methods for introducing genes into plants include Agrobacterium-mediated plant transformation, protoplast transformation, gene transfer into pollen or totipotent calli, injection into reproductive organs and injection into immature embryos. Each of these methods has distinct advantages and disadvantages. Thus, one particular method of introducing genes into a plant species may not necessarily be the most effective for another plant species.

Agrobacterium tumefaciens-mediated transfer is a widely applicable system for introducing genes into plant cells because the DNA can be introduced into whole plant tissues, bypassing the need for regeneration of an intact plant from a protoplast. The use of Agrobacterium-mediated expression vectors to introduce DNA into plant cells is well known in the art. See, for example, the methods described by Fraley et al., Biotechnology, 3:629 (1985) and Rogers et al., Methods in Enzymology, 153:253-277 (1987). Further, the integration of the T-DNA is a relatively precise process resulting in few rearrangements. The region of DNA to be transferred is defined by the border sequences and intervening DNA is usually inserted into the plant genome as described by Spielmann et al., Mol. Gen. Genet. 205:34 (1986) and Jorgensen et al., Mol. Gen. Genet., 207:471 (1987). Modern Agrobacterium transformation vectors are capable of replication in Escherichia coli as well as Agrobacterium, allowing for convenient manipulations as described by Klee et al., in Plant DNA Infectious Agents, T. Hohn and J. Schell, eds., Springer-Verlag, New York (1985) pp. 179-203. Further recent technological advances in vectors for Agrobacterium-mediated gene transfer have improved the arrangement of genes and restriction sites in the vectors to facilitate construction of vectors capable of expressing various polypeptide coding genes. The vectors described by Rogers et al., supra, have convenient multi-linker regions flanked by a promoter and a polyadenylation site for direct expression of inserted polypeptide coding genes and are suitable for present purposes.

Agrobacterium-mediated transformation of leaf disks and other tissues appears to be limited to plant species that A. tumefaciens naturally infects. Thus, Agrobacterium-mediated transformation is most efficient in dicotyledonous plants. However, the transformation of monocotyledonous plants using Agrobacterium can also be achieved. See, for example, Bytebier et al., Proc. Natl. Acad. Sci., 84:5345 (1987).

Although Agrobacterium-mediated transformation is the method of choice in those plant species where it is efficient, transformation of monocots, such as rice, corn, and wheat are usually transformed using alternative methods.

Transformation of plant protoplasts can be achieved using methods based on calcium phosphate precipitation, polyethylene glycol treatment, electroporation, and combinations of these treatments. See, for example, Potrykus et al., Mol. Gen. Genet., 199:183 (1985); Lorz et al., Mol. Gen. Genet., 199:178 (1985); Fromm et al., Nature, 319:791 (1986); Uchimiya et al., Mol. Gen. Genet., 204:204 (1986); Callis et al., Genes and Development, 1:1183 (1987); and Marcotte et al., Nature, 335:454 (1988). Application of these systems to different plant species depends upon the ability to regenerate that particular plant species from protoplasts. Illustrative methods for the regeneration of cereals from protoplasts are described in Fujimura et al., Plant Tissue Culture Letters, 2:74 (1985); Toriyama et al., Theor Appl. Genet., 73:16 (1986); Yamada et al., Plant Cell Rep., 4:85 (1986); Abdullah et al., Biotechnology, 4:1087 (1986).

To transform plant species that cannot be successfully regenerated from protoplasts, other ways to introduce DNA into intact cells or tissues can be utilized. Among these alternatives, the “particle gun” or high-velocity microprojectile technology can be utilized. Using such technology, DNA is carried through the cell wall and into the cytoplasm on the surface small metal particles with a diameter of about 1 micron that have been accelerated to speeds of one to several hundred meters per second as described in Klein et al., Nature, 327:70 (1987); Klein et al., Proc. Natl. Acad. Sci. U.S.A., 85:8502 (1988); and McCabe et al., Biotechnology, 6:923 (1988). The metal particles penetrate through several layers of cells and thus allow the transformation of cells within tissue explants. Transformation of tissue explants eliminates the need for passage through a protoplast stage and thus speeds the production of transgenic plants.

In addition, DNA can be introduced into plants also by direct DNA transfer into pollen as described by Zhou et al., Methods in Enzymology, 101:433 (1983); D. Hess, Intern Rev. Cytol., 107:367 (1987); Luo et al., Plant Mol. Biol. Reporter, 6:165 (1988). Expression of polypeptide coding genes can be obtained by injection of the DNA into reproductive organs of a plant as described by Pena et al., Nature, 325:274 (1987). DNA can also be injected directly into the cells of immature embryos and the rehydration of desiccated embryos as described by Neuhaus et al., Theor. Apl. Genet., 75:30 (1987); and Benbrook et al., in Proceedings Bio Expo 1986, Butterworth, Stoneham, Mass., pp. 27-54 (1986). DNA can also be introduced into plant cells through mixing cellular material and expression vectors with small, needle-like silicon carbide “whiskers” that are typically 0.6 microns in diameter and 10-80 microns in length (Kaeppler et al., Plant Cell Rep, 9:415 (1990).

h. Plant Regeneration

After determination of the presence and expression of the desired gene products in the transformed cells or tissues, a whole plant is regenerated. Plant regeneration can be from cultured protoplasts, or from calli or other tissues that have been transformed. The regeneration of plants from either single plant protoplasts or various explants is well known in the art. See, for example, E. B. Herman, Recent Advances in Plant Tissue Culture. Vol. 6. Regeneration and Micropropagation: Techniques, Systems and Media 1997-1999, Agritech Consultants, Shrub Oak, N.Y. (2000); and Methods for Plant Molecular Biology, A. Weissbach and H. Weissbach, eds., Academic Press, Inc., San Diego, Calif. (1988). This regeneration and growth process includes the steps of selection of transformed cells and shoots, rooting the transformed tissue and growth of the plantlets in soil.

Plant regeneration from cultured protoplasts of certain species is described in Evans et al., Handbook of Plant Cell Cultures, Vol. 1: (MacMillan Publishing Co. New York, 1983); and Vasil I. R. (ed.), Cell Culture and Somatic Cell Genetics of Plants, Acad. Press, Orlando, Vol. I, 1984, and Vol. III, 1986. All plants from which protoplasts can be isolated and cultured to give whole regenerated plants can be transformed by the present invention so that whole plants are recovered which contain the transferred gene.

Plant cells which can be transformed and regenerated into a transgenic plant capable of producing a heterologous protein of interest include dicots such as tobacco, tomato, the legumes, alfalfa, potatoes and spinach, among many others, as well as monocots such as corn, grains, oats, wheat, and barley.

3. Growth Conditions for CEA

According to the present invention, the environmental conditions under which the transgenic plant is grown are optimized to achieve maximum yield of the plant tissue and expression levels in which the heterologous protein is preferentially expressed. The CEA technology provides for optimal production of the heterologous protein in the transformed plant tissue.

CEA technology is well known in the art. For a review of CEA design, construction and management, see Dalton L. et al., Hydroponic Crop Production, NZ Hydroponics International Ltd., Tauranga, New Zealand, 1998 and Resh, H M, Hydroponic Food Production, 5^{th} Edition, Woodbridge Press, Santa Barbara, Calif., USA, 1998. CEA integrates mechanization, computer-control sensors, intensive management of nutrition and pests, and was originally developed for highly productive, high-quality crop production. Under CEA, plants are cultivated in an enclosure within which the environmental factors that are generally recognized as influencing plant growth, maturation and productivity, are systematically programmed and carefully controlled. Typically, the controlled environmental conditions include the intensity, duration and spectral distribution of illumination; humidity and flow rate of the air; atmospheric CO₂ concentration; the composition of the nutrients supplied to the growing plants; substrate water potential and substrate pH; and temperature; among others.

Hydroponic systems have been developed in parallel with CEA, and include the nutrient film technique (NFT), ebb and flood, and aerated liquid flow systems to optimize nutrition and minimize water stress. Dalton L. et al., 1998, ibid., pp.63-107. Nutrient application is limited to the amount taken up by the crop. Nutrient balance may be changed rapidly to account for differing light, humidity and crop-cycle differences.

In CEA installations in which hydroponics techniques are employed, factors relating to nutrients, such as nutrient composition and substrate temperature and pH, are most easily controlled. The nutrient solutions used with hydroponics may be analyzed for chemical composition and replenished as necessary to maintain their compositions within desired ranges.

An aerosol delivery system can also be used as the CEA system. See, for example, A. J. Cooper, Improved Film Technique Speeds Growth, The Grower, Mar. 2, 1974; Hardy Nursery Stock Production in Nutrient Film, The Grower, May 4, 1974; A. J. Cooper, Rapid Progress Through 1974 With Nutrient Film Trials, The Grower, Jan. 25, 1975. Soil? Who Needs It?, American Vegetable Grower, August & September, 1974. The nutrient film technique employs sloped tubes or troughs, commonly called gullies, in which the plant roots are contained and through which a continuous nutrient solution flow is maintained. The quantity of nutrient flow is carefully controlled and normally held at a rate such that only a small part of the root mass is contacted by the nutrient stream directly, capillary attraction or “wicking” being relied on to extend the nutrient-wetted area over and through the entire root mass. Nutrient solution that is not absorbed by the plant roots is collected and re-circulated, usually after analysis of its composition and replenishment of any deficiency.

As is well known to the skilled artisan, optimum conditions for plant growth depend on many factors. Optimum plant growth conditions vary according to the genetic make-up of the plant species involved, which tissue type(s) is to be harvested for extraction of the heterologous protein of interest, and the developmental stage of the plant.

The environmental conditions are also selected to maximize the expression of the CEA promoter that is operably linked to the heterologous gene encoding the protein of interest. According to one preferred embodiment, the heterologous gene is operably linked to a light-inducible promoter such as the promoter from the gene encoding the Rubisco small subunit protein or the chlorophyll a/b binding protein. Extended photoperiods up to continuous lighting with high illumination intensity are preferred when a light-inducible promoter is the CEA promoter. Preferred length of illumination for the present invention must be optimized for each transgenic plant species and cultivar but is at least about 8 hours, at least about 10, at least about 12 hours, preferably about 14 hours, more preferably about 16 hours, more preferably about 18 hours; more preferably about 20 hours; more preferably about 22 hours; most preferably about 24 hours. The optimum environmental conditions will depend on such factors as the genetic background of the plant and the characteristics of the CEA light-inducible promoter. Preferred illumination intensity for the present invention must also be optimized for each plant species and cultivar but generally ranges between about 200 and about 550 μE/sec/m².

The preferred atmospheric CO₂ concentration for the present invention must be optimized for each plant species and cultivar but generally ranges between about 350 to about 2,500 ppm. The preferred atmospheric CO₂ concentration for the present invention must be optimized for each transgenic plant species and cultivar. The optimum atmospheric CO₂ concentration will depend on such factors as the genetic background of the plant and the characteristics of the CEA CO₂-inducible promoter. Genes comprising CO₂-inducible promoter, for example Rubisco (rbcS) and those in Sinechococcus sp. (cyanobacteria), are known. Murchie et al., Plant Physiol Biochem 37: 251-260 (1999). Scanlan et al., Gene 90: 43-49 (1990).

The preferred temperature for the present invention must be optimized for each plant species and cultivar but generally ranges between about 20 and 40 C. The preferred temperature for the present invention must be optimized for each transgenic plant species and cultivar. The preferred temperature may comprise a temperature range that encompasses day-night variations in ambient temperature within an acceptable range for specific CEA conditions. The optimum temperature will depend on such factors as the genetic background of the plant and the characteristics of the CEA heat-inducible promoter. Genes comprising a heat-inducible promoter are known, for example, the hsp80 gene. Comai L. et al. 1993 U.S. Pat. No. 5,187,267.

Optimum growth conditions for S. tuberosum in a CEA system were found to be 24 hours per day continuous light when the plants were grown at about 24 C. Tibbitts et al., Adv. Space Res. 7: 115 (1987). These conditions can be varied to optimize heterologous protein production depending on the growth characteristics of the transgenic S. tuberosum cultivar, the plaint parts to be harvested and the characteristics of the CEA promoter.

The optimum growth conditions in CEA for S. oleracea and B. oleracea were 16 hours per day continuous light at 24 C. Both et al. Hydroponic Spinach Production Handbook 1997; Kumari et al., Indian J. Plant Physiol. 37: 142 (1994); and Bhaskar et al., J. Environ. Biol. 15: 55 (1994). These conditions can be varied to optimize heterologous protein production depending on the growth characteristics of the transgenic cultivar, the plant parts to be harvested and the characteristics of the CEA promoter.

The optimum growth conditions in CEA for B. juncea var. Czerniak were 9-10 hours per day of continuous light at about 24 C. These conditions can be varied to optimize heterologous protein production depending on the growth characteristics of the transgenic cultivar, the plant parts to be harvested and the characteristics of the CEA promoter.

Finally, the optimum growth conditions in CEA for B. oleracea var. acephala; B. oleracea var. alboglabra; B. chinensis and B. parachinensis were at least 20 hours per day (will grow anywhere between 8-24) and optimally between 12 and 21 C. (will grow between 4-30 degrees C.). Paul, Bangladesh J Bot 20:143 (1991). Hodges et al., Culture of Cole Crops, Paper G92-1084, U. Nebraska, Lincoln, (1992).

4. Protein Stability

The stability of heterologous proteins within plant tissues, and upon extraction from transgenic plants, dramatically affects yield of the protein of interest. It has been observed that chimeric genes wherein a DNA sequence encoding a targeting sequence is operably linked to the structural gene produce a fusion protein that is directed for co-translational insertion into the endoplasmic reticulum, thereby increasing the stability of fusion protein within transgenic plants. See U.S. Pat. No. 5,959,177. Similar fusion protein stability increases have been observed in our own laboratory for a DNA sequence encoding a targeting sequence that is operably linked to the structural gene producing a fusion protein that is directed for co-translational insertion into the chloroplast. Dai Z. et al., Mol. Breeding, 6:277-285 (2000). In the absence of a targeting sequence, the heterologous protein recovery can be very low.

In general it is prudent to include protease inhibitors within the extraction cocktails in order to maximize protein recovery from transgenic plant tissues. Cost-effective production of transgenic proteins, however, requires simplicity. Accordingly, it is advantageous to select plant species or cultivars for the CEA system that exhibit low rates of degradation of the protein or peptide of interest.

The selection method is designed to identify plants for transformation and heterologous protein production based on stability of the protein in plant extracts. Selection of plants for use in the CEA system that have plant extracts in which a heterologous protein is stable should increase the amount of heterologous protein that can be recovered from plant extracts during the heterologous protein purification process.

In general, the stability of a protein added to plant extracts is determined to select those plants that are best suited for heterologous protein production. More specifically, the stability of the heterologous protein to be expressed in the transgenic plant is determined. Plant extracts are made from plants of the age from which heterologous protein will be extracted during the commercial protein production. Additionally, plant extracts are made from the plant part, such as leaf material, that will be harvested during commercial protein production.

According to one embodiment of the invention, protein stability is measured by (1) preparing a suitable tissue extract wherefrom the heterologous protein of interest is to be extracted; (2) spiking the suitable tissue extract with a protein, such as the human coagulation Factor VIII protein, and (3) measuring the concentration and/or activity of the spiked protein at different time intervals under normal isolation and purification conditions for the protein. The spiked protein should remain stable in the tissue extract according to the instant invention, that is, no significant degradation or loss of activity should be observed of the spiked protein in a time period necessary for the heterologous protein to be isolated and purified. Plant species or cultivars are selected for the CEA system that exhibits low rates of degradation of the protein or peptide of interest.

5. Protein Isolation and Purification

Processes for isolating proteins, peptides and viruses from plants have been described in the literature (Johal, U.S. Pat. No. 4,400,471, Johal, U.S. Pat. No. 4,334,024, Wildman et al., U.S. Pat. No. 4,268,632, Wildman et al., U.S. Pat. No. 4,289,147, Wildman et al., U.S. Pat. No. 4,347,324, Hollo et al., U.S. Pat. No. 3,637,396, Koch, U.S. Pat. 4,233,210, and Koch, U.S. Pat. No. 4,250,197. The succulent leaves of plants, such as tobacco, spinach, soybean, and alfalfa, are typically composed of 10-20% solids, the remaining fraction being water. The solid portion is composed of a water soluble and a water insoluble portion, the latter being predominantly composed of the fibrous structural material of the leaf. The water soluble portion includes compounds of relatively low molecular weight (MW), such as sugars, vitamins, alkaloids, flavors, amino acids, and other compounds of relatively high MW, such as natural and recombinant proteins.

Proteins in the soluble portion of the plant tissue can be further divided into two fractions. One fraction comprises predominantly a photosynthetic enzyme, Rubisco. The Rubisco enzyme has a molecular weight of about 550 kD. This fraction is commonly referred to as “fraction 1 protein.” Rubisco is abundant, comprising up to 25% of the total protein content of a leaf and up to 10% of the solid matter of a leaf. The other fraction contains a mixture of proteins and peptides have molecular weights typically ranging from about 3 kD to about 100 kD and other compounds including sugars, vitamins, alkaloids and amino acids. This fraction is collectively referred to as “fraction 2 proteins.” Fraction 2 proteins can be native host materials, heterologous proteins and peptides. Transgenic plants may also contain plant virus particles having a molecular size greater than 1,000 kD.

The basic process for isolating plant proteins generally begins with disintegrating leaf tissue and pressing the resulting pulp to produce a raw plant extract. The process is typically performed in the presence of a reducing agent or antioxidant to suppress undesirable oxidation. The raw plant extract, which contains various protein components and finely particulate green pigmented material, is pH adjusted and heated. The typical pH range for the raw plant extract after adjustment is between about 5.3 and about 6.0. This range has been optimized for the isolation of fraction 1 protein. Heating, which causes the coagulation of green-pigmented material, is typically controlled near 50° C. The coagulated green-pigmented material can then be removed by moderate centrifugation to yield a secondary plant extract. The secondary plant extract is subsequently cooled and stored at a temperature at or below room temperature. After an extended period of time, e.g. 24 hours, Rubisco is crystallized from the brown juice. The crystallized fraction 1 protein can subsequently be separated from the liquid by centrifugation. Fraction 2 proteins remain in the liquid, and they can be purified upon further acidification to a pH near 4.5. Alternatively, the crystal formation of Rubisco from secondary plant extract can be induced by adding sufficient quantities of polyethylene glycol (PEG) in lieu of cooling.

According to one embodiment of the invention, the transgenic plant produces at least 100 kg heterologous protein/acre/year under the continuous production system of the CEA. According to another embodiment, the plant system produces at least 150 kg heterologous protein/acre/year under the continuous production system of the CEA. In a preferred embodiment, the transgenic plant produces at least 200 kg heterologous protein/acre/year under the continuous production system of the CEA. More preferably, the transgenic plant produces at least 250 kg heterologous protein/acre/year under the continuous production system of the CEA. Particularly preferable is a plant system that produces at least 300 kg heterologous protein/acre/year under the continuous production system of the CEA. Most preferable is a plant system that produces up to 1200 kg/acre/year.

The following examples are given to illustrate the present invention. It should be understood, however, that the invention is not to be limited to the specific conditions or details described in these examples. Throughout the specification, any and all references to publicly available documents are specifically incorporated by reference.

### EXAMPLES

### Example 1

Agrobacterium-Mediated Transformation of S. tuberosum

S. tuberosum plants of cultivar FL1607 were regenerated under aseptic conditions for transformation with an expression vector in which a light-inducible promoter was operably linked to a heterologous promoter. The light-inducible promoter was from the tomato small subunit Rubisco gene. Pichersky et al., Proc Natl Acad Sci USA 82: 3880-3884 (1986). Carrasco et al. Plant Mol. Biol. 21:1-15 (1993). S. tuberosum single-node stem segments were excised and placed in culture under the conditions described below. Explants used to initiate in vitro culture were sterilized using 5% (v/v) sodium hypochlorite bleach solution and rinsed 5 times with sterile deionized water prior to cultivation. All sterile cultures were maintained on solid medium containing 200 mg/L carbenicillin. Basal medium consisted of the salts recommended by Murashige and Skoog supplemented with 100 mg/L myo-inositol, 3% sucrose and 0.4 mg/L thiamine-HCl and solidified with 0.8% (wlv) Phytoagar (GIBCO Life Technologies).

Shoots possessing adventitious roots at the lower nodes developed from the axillary buds of those single-node stem segments. The middle 3 to 5 single-node stem segments from these shoots were serially sub-cultured every 3-4 weeks. Five single node stem segments were placed in GA-7 vessels (Magenta) containing 40 ml of basal medium supplemented with 60 mM sucrose and incubated at 25° C. under diffuse fluorescent light (from equal numbers of cool-white and Grow-lux [Sylvania] lamp, energy flux approx. 10 Wm⁻²) for 16 h, alternating with 8 h of darkness. Basal medium consisted of the salts recommended by Murashige and Skoog supplemented with 100 mg/L myo-inositol and 0.4 mg/L thiarmine-HCl and solidified with 0.8% (wlv) Phytoagar (GIBCO Life Technologies).

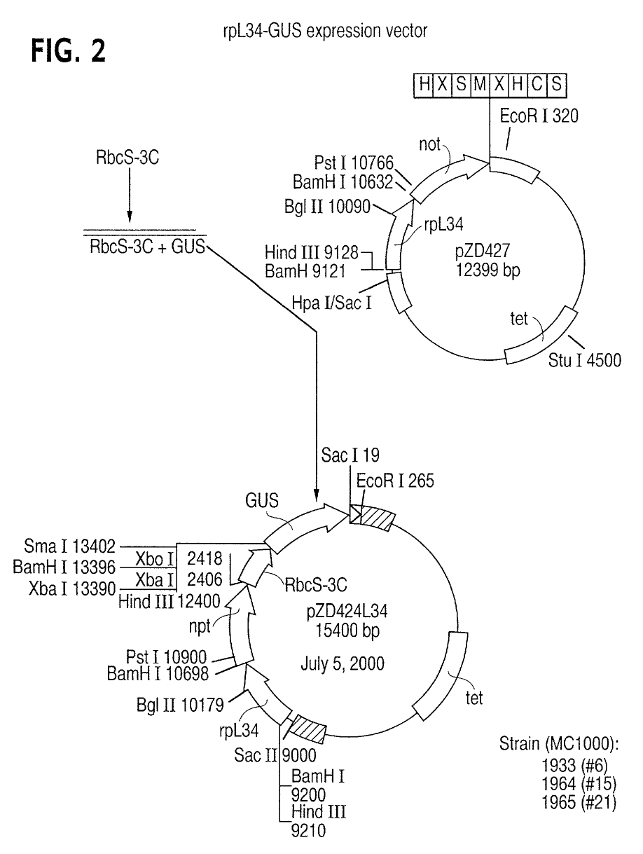

A. tumefaciens strain LBA4404 was grown in tubes containing 2 ml of sterile YEP medium which was composed of 10 g/L yeast extract, 10 g/L peptone, and 5 g/L NaCl and adjusted to pH 7.0 before sterilization. After autoclaving, the medium was supplemented with filter-sterilized solutions of kanamycin sulfate and tetracycline to a final concentration of 10 and 5 mg/L, respectively. The tubes were placed near horizontal in a rotary wheel spinning at 180 rpm and incubated at 280 C. for 15-20 h until the bacteria reached late log phase (>10⁹ bacteria/mL). Strain LBA4404 harbors a vector designated pZD424 comprising the promoter from the Rubisco small subunit gene operably linked to the GUS gene. Additionally, pZD424comprises the promoter from the Agrobacterium tumefaciens nopaline synthetase (nos) operably linked to the neomycin phosphotransferase II (npt II) gene from the bacterial transposon Tn5. Alternatively, pZD424L34, shown in FIG. 2, comprises the promoter from the tobacco ribosomal protein gene (rpL34) operably linked to the neomycin phosphotransferase II (npt II) gene from the bacterial transposon Tn5.

Segments of stem internode measuring about 8 -10 mm long were excised under aseptic conditions from the first two internodes taken from the top of 4-5-week old sterile cultured plants. The internode explants were placed on 100×25 mm Petri plates containing 30 ml of stage I medium (basal medium supplemented with 60 mM sucrose, 10 mg/L gibberellic acid, 200 μg/L naphthaleneacetic acid and 2.24 mg/L benzylaminopurine) and incubated for 4 days at 23° C. with a 16 h/day photoperiod. Following this pre-treatment, 50 internode segments were placed in a sterile Petri dish containing suspensions (diluted 1:100 with sterile water) of a saturated liquid culture of A. tumefaciens expression vector pZD424 and co-cultivated at 25° C. for 15 min. After removing excess liquid by blotting on 3M filter papers, up to 50 internode explants were returned to plates of stage I medium and incubated under the conditions described above until a slight bacterial ring developed at the cut-edge surfaces of the explant (2-3 days). The explants were washed with MS medium containing 250 mg/L cefotaxime (purchased from local hospital) three times. The excess MS liquid was removed by blotting the internode segments on 3M filter paper and then placed in Magenta GA-7 vessels containing 40 ml of stage I medium and supplemented with 250 mg/L cefotaxime and 50 mg/L. kanamycin sulfate. The antibiotics were filter-sterilized and added to the medium after autoclaving. The explants were then incubated for 15 to 20 days as described above.

To produce presumptively transformed shoots, up to 12 explants were placed in GA-7 vessels containing 40 ml of Stage II medium. Stage II medium was the same as the stage I medium minus the auxin, but supplemented with both antibiotics.

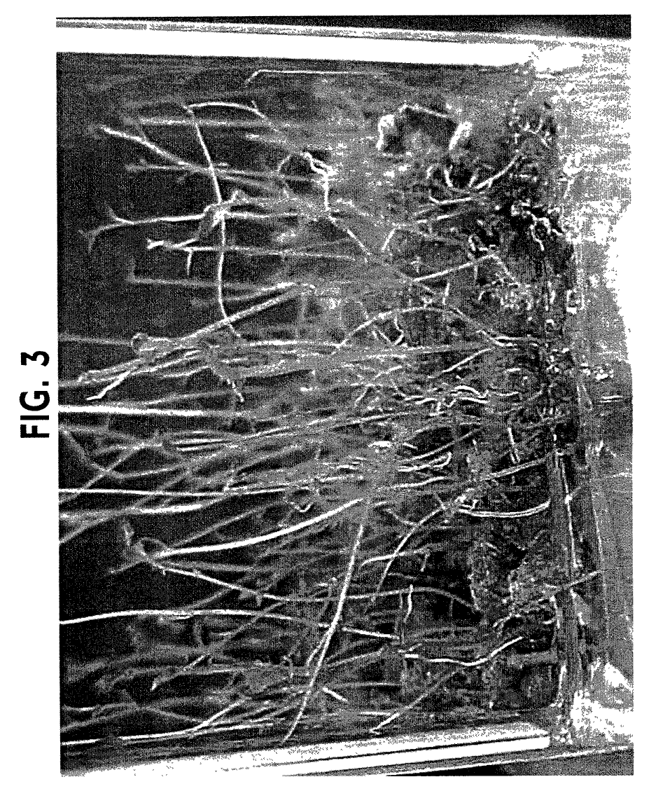

Using this protocol, an average transformation frequency of 1000% (i.e., 10 positive transformants per 1 potato stem internode explant). Pictorial examples suggesting this transformation frequency are shown in FIG. 3. It should be noted that transformation frequency data were calculated based on the number of rooting shoots observed grown on antibiotic-based selection medium, in the absence of auxin and not merely upon the number of shoots arising from single explants grown in stage I medium.

### Example 2

Production of E1 endoglucanase Protein in the S. tuberosum in a CEA System

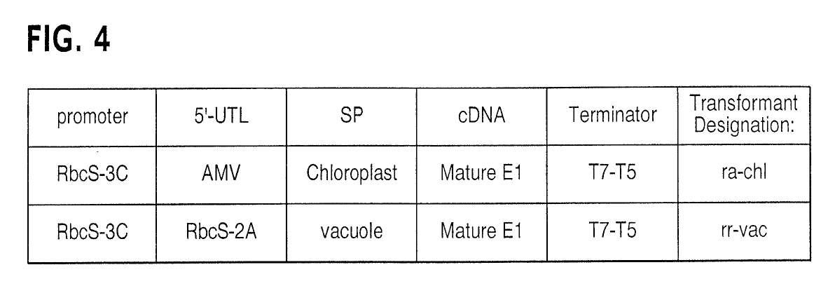

Optimization of Acidothermus cellulolyticus endoglucanase (E1) gene expression in transgenic potato (Solanum tuberosum L.) made from cultivar FL1607 was examined where the E1 coding sequence was operably linked to the leaf-specific tomato RbcS-3C promoter. Plasmid pPMT4-5 containing the endoglucanase (E1) gene was isolated from an A. cellulolyticus genomic library. A 1562 bp fragment containing the mature peptide coding region was isolated from pPMT4-5 by PCR, where PCR conditions were described previously. Dai et al. Appl Biochem Biotech 77-79:689-699 (1999). In order to fuse the mature E1 coding sequence in frame to the sequence of a proper transit signal peptide, an adapter was introduced at the 5′-end of the mature E1 coding sequence by PCR. Two signal peptide sequences used in this study were the sporamin signal peptide (Matsuoka et al. J Cell Biol 130: 1307-1318 (1995)) and the Rubisco small subunit RbcS-2A signal peptide (Park et al. Plant Mol Biol 37: 445-454 (1998)). In some instances the AMV untranslated leader (UTL) was fused to the 3′ end of the RbcS-3C promoter. The fragment containing the signal peptide and E1 coding sequence was fused in frame downstream of the Rubisco small subunit RbcS-3C promoter or the RbcS-3C promoter/AMV 5′ UTL (FIG. 4). The proper fusion of DNA fragments between the promoter, signal peptide, and E1 coding sequence was verified by DNA sequencing.

Transgenic potato plants were obtained by the co-cultivation method using potato leaf strips grown aseptically on Murashige and Skoog (MS) agar supplemented with 60 mM sucrose and appropriate amounts of plant growth regulators. All transformants were grown under a 14 h light (25-28° C., 60% relative humidity)/10 h dark (22° C., 70% relative humidity) cycle. Irradiance, provided by six high-pressure metal halide lamps (Philips, USA) was 350 to 500 μmol quanta m⁻² s⁻¹ at the plant canopy.

The third or fourth healthy leaf from the shoot apex of transgenic potato plants grown for 4 weeks in the growth room were harvested for E1 enzyme extraction. Leaf tissues were sectioned into 1 cm² leaf discs and pooled. Approximate 0.1 g of leaf discs was used for E1 enzyme extraction with a pellet pestle (Kontes Glass Co, Vineland, N.J.) in a microcentrifuge tube and 4 volumes of ice-cold extraction medium. The extract medium contained 80 mM MES, pH 5.5, 10 mM β-mercaptoethanol, 10 mM EDTA, pH 8.0, 0.1% sodium N-lauroyl sarcosinate, 0.1% Triton X-100, 1 mM PMSF, 10 μM Leupeptin, and 1 μg mL⁻¹ each of aprotinin, pepstin A, and chymostatin. The supernatant from crude extract centrifuged at 15,000 g and 4° C. for 10 min was used for protein determination, enzymatic analysis, polyacrylamide gel electrophoresis, and Western blot analyses. The concentration of soluble protein was determined by the method of Bradford with BSA as the standard. For E1 protein extraction from potato tubers, about 0.2 to 0.3 g of tuber slices were ground with a mortar and pestle in enzyme extraction medium as described above.

The E1 enzyme reaction was conducted at 55° C. with reaction mixture containing 80 mM MES, pH 5.5, 1 mM EDTA, 1 mM DTT, and 5 to 10 μL of enzyme extract in a final volume of one mL. The enzyme reaction was initiated by adding 2 mM 4-methylumbelliferone-β-D-cellobioside (MUC) into the reaction mixture. Hundred microliter aliquots was removed at 15, 30, and 45 min intervals and put into 1.9 mL 0.2 M Na₂CO₃ buffer to terminate the reaction. The fluorescent reporter moiety, 4-methylumbelliferone (MU), released from 4-MUC by the action of E1, has a peak excitation of 365 nm (UV) and a peak emission of 455 nm (blue). Emission of fluorescence from the mixture was measured with a Hoefer DyNA Quant 200 Fluorometer (Hoefer Pharmacia Biotech, San Francisco, Calif.) using 365 nm excitation and 455 nm emission filters, respectively. Enzyme activities were expressed on a total leaf soluble protein basis or fresh weight basis.

Electrophoresis analysis of protein extracts was performed in a 7.5 to 15% (w/v) linear gradient polyacrylamide gel containing 0.1% SDS and stabilized by a 5 to 17% (w/v) linear sucrose gradient or 4 to 20% (w/v) precast mini gel (Bio-Rad laboratories, Hercules, Calif.) as described previously. Dai et al. ibid (1999). The E1 protein separated by electrophoresis was then electrophoretically transferred onto a nitrocellulose membrane (BA-S85; Schleicher & Schuell, Keene, N.H.). The protein was reacted with affinity-purified mouse monoclonal antibody raised against full-length E1 protein (in 1:250 dilution). The antibody was detected using a Immun-Blot Assay Kit (BIO-RAD, Hercules, Calif.) and a goat anti-mouse secondary antibody (IgG) conjugated with alkaline phosphatase (Pierce, Rockford, Ill.). The E1 protein used as a positive control in these experiments was purified from culture supernatant of Streptomyces lividans carrying a plasmid containing a 3.7 kb genomic fragment of A. cellulolyticus E1 gene.

The amount of E1 expressed in leaf tissues was estimated by densitometry analysis. Protein blot bands were scanned with a Hewlett Packard ScanJet 6100C Scanner (Hewlett Packard Inc, Palo Alto, Calif.). The imaging data were then analyzed with the DENDRON 2.2 program (Solltech Inc Oakdale, Iowa). A series of diluted E1 proteins (known amounts) from S. lividans expression was used as a standard for estimating E1 accumulation in transgenic plants.

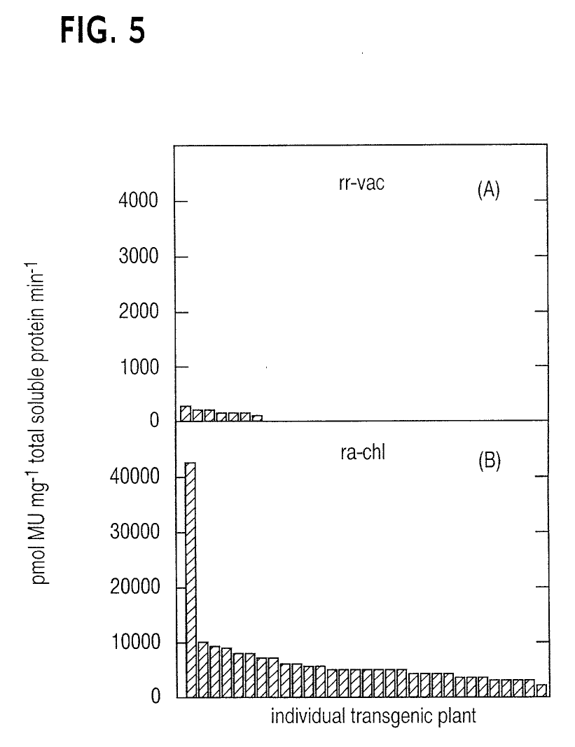

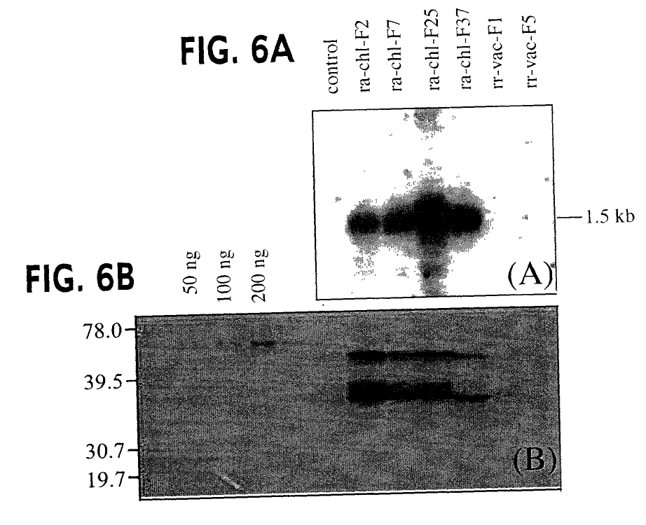

Average E1 activity in leaf extracts of potato transformants, where E1 protein was targeted by the chloroplast signal peptide was much higher than that of E1 targeting by the vacuole signal peptide (FIG. 5). E1 protein accumulated up to 2.6% of total leaf soluble protein, where the E1 gene was under control of the RbcS-3C promoter, alfalfa mosaic virus 5′-untranslated leader, and RbcS-2A signal peptide. Based on average E1 activity and E1 protein accumulation in leaf extracts, E1 protein production is higher in potato than in transgenic tobacco bearing the same transgene constructs reported in Dai et al. Transgenic Res 9: 43-54 (2000). Results from E1 activity measurements, protein immunoblotting and RNA gel-blot analyses showed that E1 expression under the control of RbcS-3C promoter was specifically localized in leaf tissues (FIG. 6).

### Example 3

Production of E1 endoglucanase Protein in S. tuberosum in a CEA System

Transgenic potato plants expressing E1 were obtained as described in example 2.

“T1” plants were raised from propagules of two original transformants (1319-7 and 1319-24) originated by vegetative propagation from tubers. These plants were initially grown under a 12 h light (25-28° C., 60% relative humidity)/12 h dark (22° C., 70% relative humidity) cycle with irradiance provided by three high-pressure metal halide lamps (Philips, USA) at 350 to 500 μmol quanta m⁻² s⁻¹ at the plant canopy. After two weeks, half of the plants from each line (1319-7 and -24) were transferred to a separate growth chamber and grown under 24 h light (25-28° C., 60% relative humidity) with irradiance provided by three high-pressure metal halide lamps at 500 μmol quanta m⁻² s⁻¹ at the plant canopy. The remaining “baseline” plants were grown under the original 12 h light/12 h dark conditions as specified previously.

The third or fourth healthy leaf from the shoot apex of transgenic potato plants grown for two weeks and four weeks in individual chambers was harvested for E1 enzyme extraction. Leaf tissues were sectioned into 1 cm² leaf discs and pooled. Approximate 0.1 g of leaf discs was used for E1 enzyme extraction with a pellet pestle (Kontes Glass Co, Vineland, N.J.) in a microcentrifuge tube and 4 volumes of ice-cold extraction medium. The extract medium contained 80 mM MES, pH 5.5, 10 mM β-mercaptoethanol, 10 mM EDTA, pH 8.0, 0.1% sodium N-lauroyl sarcosinate, 0.1% Triton X-100, 1 mM PMSF, 10 μM Leupeptin, and 1 μg mL⁻¹ each of aprotinin, pepstin A, and chymostatin.

The E1 enzyme reaction was conducted at 55° C. with reaction mixture containing 80 mM MES, pH 5.5, 1 mM EDTA, 1 mM DTT, and 5 to 10 μL of enzyme extract in a final volume of one mL. The enzyme reaction was initiated by adding 2 mM 4-methylumbelliferone-β-D-cellobioside (MUC) into the reaction mixture. Hundred microliter aliquots was removed at 15, 30, and 45 min intervals and put into 1.9 mL 0.2 M Na₂CO₃ buffer to terminate the reaction. The fluorescent reporter moiety, 4-methylumbelliferone (MU), released from 4-MUC by the action of E1, has a peak excitation of 365 nm (UV) and a peak emission of 455 nm (blue). Emission of fluorescence from the mixture was measured with a Hoefer DyNA Quant 200 Fluorometer (Hoefer Pharmacia Biotech, San Francisco, Calif.) using 365 nm excitation and 455 nm emission filters, respectively. Enzyme activities were expressed on a total leaf soluble protein basis or fresh weight basis.

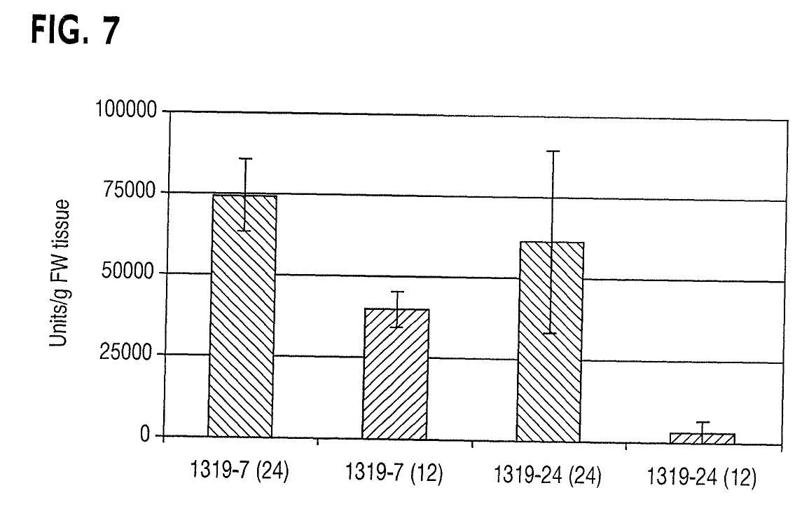

Table 1 and FIG. 7 show experimental measurements of cellulase activity resulting from 12- and 24-hour light conditions. For plant line 1319-7, increases in cellulase activity in plants grown under 24-hour light over the two week time period were on average 90% higher than those of 12-hour light control plants. More dramatically, plant line 1319-24 under 24-hour light conditions showed an increase in activity 20-fold of that of 12-hour light control plants. Table 2 shows expression level increases (in % total soluble protein) under 24-hour light and 12-hour light conditions. Similar to cellulase activity data, plants grown under 24 hour light show an increase of 1% TSP over the two week growth period, as compared to control plants that show an increased of only 0.46% TSP.

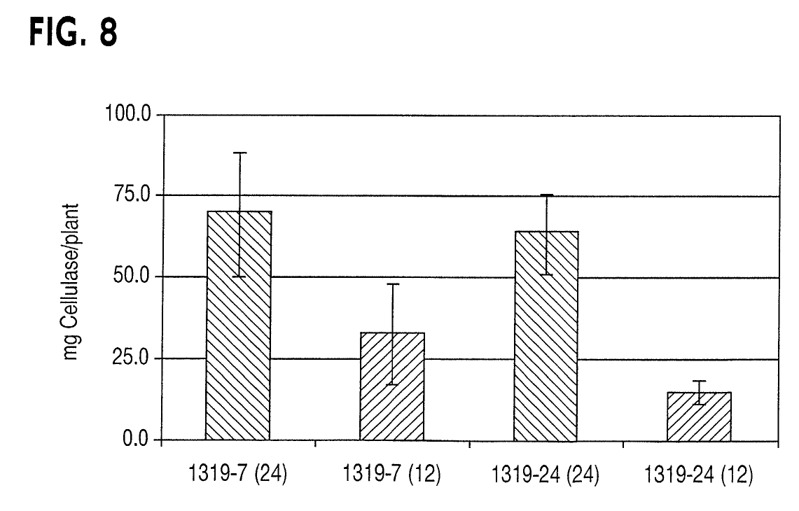

After four weeks in separate growth chambers, all plants were harvested and total fresh weight of potato tops (foliage, stems and branches) was measured. Levels of E1 cellulase production were subsequently calculated from E1 activity measurements and FW of plant green tissues. This information is shown in FIG. 8. The data clearly demonstrate greater levels of cellulase production from plant lines cultivated under a continuous photoperiod. Plant lines 1319-24 and 1319-7, respectively, showed 323% and 112% increases in cellulase production under continuous photoperiod over plants from the same lines grown under a 12 hour light-dark cycle.

TABLE 1
Cellulase (MUG) activity in transgenio potato leaf tissues from
plants grown under 12- and 24-hour photoperiods.

| | | | | |
|---|---|---|---|---|
| | | | | Std. |
| Day 0 | Day 14 | Change | Average | Dev. |

Line 1319-7

| 24 hr light | MUG units/g FW tissue | | | | |
|---|---|---|---|---|---|
| 1319-7-1 | 23176. | 90809.4 | 67633.2 | | |
| 1319-7-4 | 9779. | 96793.5 | 87013.8 | | |
| 1319-7-7 | 8425. | 75666.0 | 67240.9 | | |
| | | | | 73962.6 | 11304.3 |
| 12 hr light | | | | | |
| 1319-7-2 | 7135. | 49606.7 | 42471.1 | | |
| 1319-7-3 | 16304. | 55819.9 | 39515.8 | | |
| 1319-7-5 | 11090. | 42879.0 | 31788.1 | | |
| 1319-7-6 | 7145. | 51831.1 | 44685.3 | | |
| | | | | 39615.1 | 5631.3 |
| Line | | | | | |
| 1319-24 | | | | | |
| 24 hr light | MUC units/g FW tissue | | | | |
| 1319-24-1 | 13696. | 97520.4 | 83823.7 | | |
| 1319-24-2 | 7110. | 36018.6 | 28908.5 | | |
| 1319-24-3 | 10609. | 79626.7 | 69017.0 | | |
| | | | | 60583.1 | 28412.4 |
| 12 hr light | | | | | |
| 1319-24-4 | 5974. | 11251.8 | 5277.5 | | |
| 1319-24-5 | 14975. | 15811.2 | 836.2 | | |
| | | | | 3056.9 | 3140.5 |

TABLE 2
Expression level of cellulase in % of total soluble protein

| | E1 expression level % TSP | | |
|---|---|---|---|
| | (calculated) | | |
| | 6/8/01 | 6/22/01 | Change |
| 24 hrs light | | | |
| 1319-7-1 | 1.34 | 1.79 | |
| 1319-7-4 | 0.87 | 2.92 | |
| 1319-7-7 | 0.78 | 1.63 | |
| Average | 1.00 | 2.12 | 1.12 |
| Std. Dev. | 0.30 | 0.70 | |
| 12 hrs light | | | |
| 1319-7-2 | 0.72 | 1.59 | |
| 1319-7-3 | 1.08 | 1.31 | |
| 1319-7-5 | 0.99 | 1.29 | |
| 1319-7-6 | 0.65 | 1.10 | |
| Average | 0.86 | 1.32 | 0.46 |
| Std. Dev. | 0.21 | 0.20 | |

### Example 4

Continuous Production of Recombinant Target Protein in the S. tuberosum CEA System

S. tuberosum cultivar FL1607 plants transformed with pZD424 are prepared according to Example 1.

Production plants are cultivated in large greenhouses, for example multiple Arch Series 6500 greenhouse modules measuring 42×120×8 feet manufactured and constructed by the International Greenhouse Company, Seattle, Wash. Each greenhouse module includes a hydroponic (fertigation) system. The transgenic plants are currently grown using a simple “flood and drain” fertigation technique in a hydroponic solution containing 1 tsp. Osmocote Miracle Grow granules (The Scotts Company, Marysville, Ohio) per gallon of deionized water. Transgenic plants are also cultivated using the Nutrient Film Technique (NFT) in an NFT gully arrangement. Dalton L. et al., 1998, ibid., pp.80-81. Items used for fertigation and NFT systems are purchased from CropKing Incorporated, Commercial Hydroponics Division, Seville, Ohio. Plants transformed on day 0 are screened on selective medium and via PCR for proper transformation (gene insertion) and subsequently moved into a greenhouse at day 90. Between day 90 and 150 the plants are screened for expression level and favorable growth characteristics. At day 150, a single plant or plants exhibiting the highest recombinant protein expression and best growth characteristics within the population of primary transformants is selected.

Meristematic tissues from the single transformant or multiple transformed plants are harvested, propagated by cuttings to raise up approximately 33000 propagules/week within thirty weeks. Cultivation may be completed on hormone free solid medium based on Murishige and Skoog (MS) salts and associated micronutrients without growth hormones or alternatively in soil using a root initiation agent such as Rootone (0.20% 1-naphthaleneacetamide, Green Light Co., San Antonio, Tex., USA), using a 14 hour/day photoperiod of 400 umol/s/m\hat{0}2 light and 20° C. Callus initiation is avoided to eliminate any somatic variation in resulting propagules. At day 360, propagules are moved into the hydroponic greenhouse.

Approximately 16500 plants/batch will enter recombinant protein production greenhouses, yielding an overall productivity of 280 kg raw (pre-extraction and purification) recombinant protein per year. The remaining 16500 plants/batch will either be used for cutting-based propagation of plants or be sent to potato seed producers in order to maintain the transgenic plant line via potato “seed” (i.e., tubers) planting beyond the first year of full production operations. At least 30 weeks will be required in order to establish potato seed. Techniques involving seed (tuber) production and planting are well known in the art.

The operational basis of the production greenhouse is 100 kg/year of transgenic protein downstream of purification process per year, processed in 50 batches, harvested every 7 days with two weeks down time per year. Protein recovery is estimated in a downstream material balance module for individual unit processes in the separation/purification/forrnulation process train. Cumulative recovery is calculated at approximately 36% of CEA-based transgenic protein production.

Transgenic plants in the production greenhouse are grown to favor vine growth and maximum expression of the Rubisco gene promoter that is operably linked to the GUS gene. Transgenic plants are grown with 24 hours of light per day, with a light intensity 400 umol/s/m² and a temperature of 24° C. The transgenic plants are grown using variable spacing to accommodate maximum use of lighting, starting in 4 inch diameter pots at approximately 9 plants/ft² with sufficient spacing to accommodate 1.5 ft centers and 0.44 plant/ft² at harvest maturity. The potato vines are harvested starting at day 420 for the first batch, 60 days after transfer to the greenhouse. Expression levels at harvest average 3% total soluble protein for all green tissues. The yield of raw recombinant GUS protein is approximately 280 kg per total progeny (350 kg/acre/yr) propagated from the single plant or multiple plants selected at day 150. Assuming approximately 65% losses associated with harvest and downstream purification of recombinant product, the total manufacturing facility output is 100 kg/yr using approximately 35000 ft² (0.8 acre) of greenhouse floor space. At day 725, one year beyond initiation of production greenhouse operations, all plants are initiated using seed potatoes rather than propagules to avoid additional cost associated with cutting-based propagation.

### Example 5

Agrobacterium-Mediated Transformation of Mustard, Kale, Chinese Cabbage and Collards

Seeds of mustard (Brassica juncea), kale (Brassica oleracea L. cv. acephala), chinese cabbage (Brassica chinensis L.) and collards (Brassica oleracea L. cv. viridis) were obtained from the commercial seed companies. Hypocotyl segments and petioles from cotyledons were isolated from 5-day-old axenically grown seedlings (50-80 seedlings per transformation). All in vitro plant tissue cultures were grown at 25° C. in 16 hours of light followed by 8 hours of darkness.

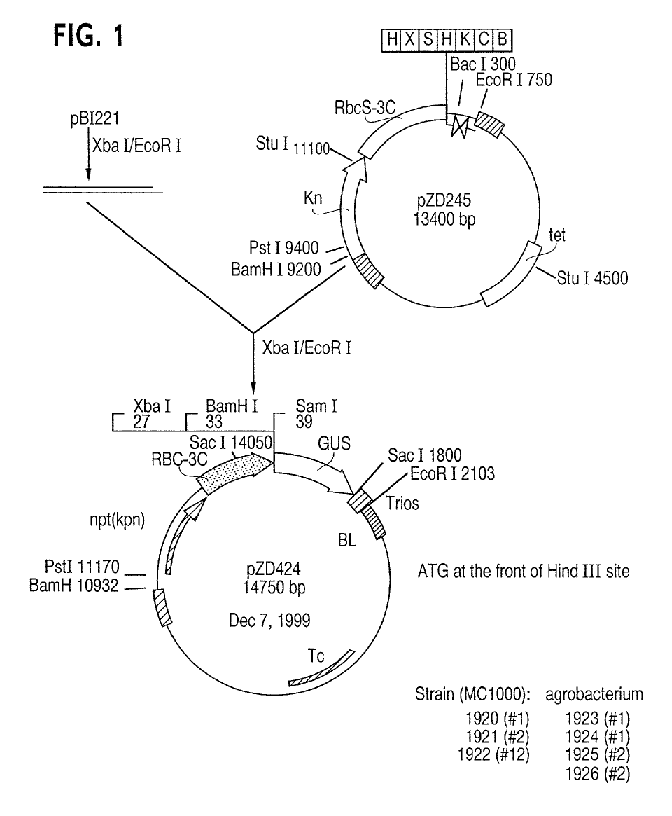

Explants were cultured for 2 days on a regeneration medium containing MS macro- and microelements and vitamins, 2 mg/L 6-benzylaminopurine (BAP), 0.05 mg/L α-naphthaleneacetic acid (NAA), 30 g/L sucrose and 7 g/L agar buffered to pH 5.8 before co-cultivation with A. tumefaciens strain C58 harboring expression vector pMP90. Expression vector pMP90 was modified to create pZD424 (FIG. 1) which comprises the promoter from the tomato Rubisco gene (RbcS-3C) operably linked to the B-glucoronidase (GUS) gene. Expression vector pZD424 also contains the promoter from the A. tumefaciens nopaline synthetase gene operably linked to the nptII gene. Alternative expression vectors also contain the tomato RbcS-3C gene promoter operably linked to the GUS gene; however the nptII selectable marker gene is operably linked to the tobacco rpL34 promoter (pZD424L34, FIG. 2).

Cotyledonary petioles were embedded in the agar medium and hypocotyls were placed on the surface of the medium in 100×15 mm petri dishes. Ten to 15 explants were cultured per plate. From 80 to 150 explants were used for each treatment, with three or four replications per treatment. All explants were cultured for a period of 2 days in darkness at 22° C.

The segments were immersed for 15 minutes in a suspension of the A. tumefaciens strain C58 harboring expression vector pZD424. A. tumefaciens strain C58 harboring expression vector pZD424 was grown to a density of A600=0.63 in YEP medium. The bacteria were previously grown for 1 d at 28° C. in liquid YEP medium in the presence of 200 μM acetosyringone (3,5-dimethoxy-4-hydroxy-acetophenone; Fluka), 10 mg/L kanamycin, and 3 mg/L tetracycline.

After immersion in the bacterial suspension, the hypocotyls and petioles were blotted dry (with 3M blot paper) and transferred to 3M filter paper covering medium containing MS salts and vitamins (M5519, Sigma), 7 g/L agarose, 10 g/L sucrose, glucose, and mannitol, 200 μM acetosyringone, 2 mg/L 6-benzylaminopurine, and 0.05 mg/L naphthalene acetic acid.

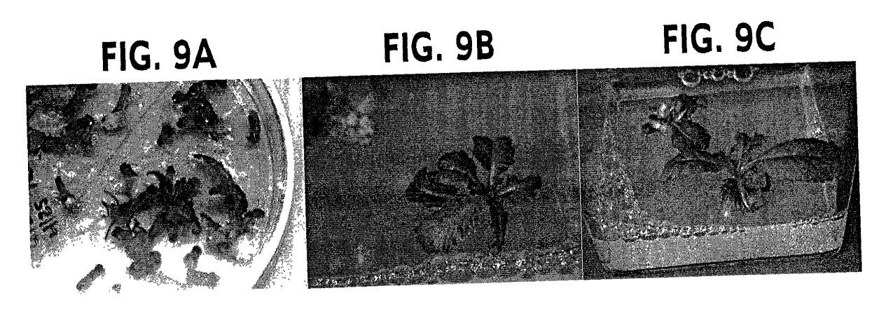

After 2 days of cultivation the hypocotyls and petioles were washed 3 times in standard liquid MS medium, blotted dry, and transferred to medium containing MS salts and vitamins, 7 g/L agarose, 10 g/L sucrose, glucose, and mannitol, 250 mg/L cefotaxime, 20 mg/L kanamycin, 2 mg/L 6-benzylaminopurine, 0.05 mg/L naphthalene acetic acid, and 30 μM AgNO3. After 10 days the hypocotyls and petioles were transferred to the same medium containing 10% coconut water. Established shoots were transferred to standard Murashige and Skoog medium containing 30 g/L sucrose, 200 mg/L cefotaxime to promote root formation. Positive mustard transformants grown on rooting medium are shown in FIG. 9.

### Example 6

Continuous Production of Recombinant Target Protein in the B. juncea CEA System

B. juncea L. cv. Czerniak (Florida Broadleaf and Southern Curled mustard) plants are transformed with appropriate expression vectors are transformed with pZD424 as described in Example 5.

Production plants are cultivated in large greenhouses, for example multiple Arch Series 6500 greenhouse modules measuring 42×120×8 feet manufactured and constructed by the International Greenhouse Company, Seattle, Wash. Each greenhouse module includes a hydroponic (fertigation) system. The transgenic plants are currently grown using a simple “flood and drain” fertigation technique in a hydroponic solution containing 1 tsp. Osmocote Miracle Grow granules (The Scotts Company, Marysville, Ohio) per gallon of deionized water. Transgenic plants are also cultivated using the Nutrient Film Technique (NFT) in an NFT gully arrangement. Dalton L. et al., 1998, ibid., pp.80-81. Items used for fertigation and NFT systems are purchased from CropKing Incorporated, Commercial Hydroponics Division, Seville, Ohio.

Plants transformed on day 0 are screened on selective medium and via PCR for proper transformation (gene insertion) and subsequently moved into a greenhouse at day 90. Between day 90 and 150 the plants are screened for expression level and favorable growth characteristics. At day 150, a single plant or plants exhibiting the highest recombinant protein expression and best growth characteristics within the population of primary transformants is selected. Meristematic tissues from the single transformant or multiple transformed plants are harvested and propagated using tissue culture methods to raise approximately 60000 propagules/week within 30 weeks. Cultivation is completed on hormone free solid medium based on Murishige and Skoog (MS) salts and associated micronutrients without growth hormones or alternatively in soil using a root initiation agent such as Rootone (0.20% 1-naphthaleneacetamide, Green Light Co., San Antonio, Tex., USA), using a 10 hour/day photoperiod of 400 umol/s/m2 light and 24° C. Callus initiation is avoided to eliminate any somatic variation in resulting propagules. At day 360, propagules are moved from tissue culture facilities into the hydroponic greenhouse One batch consists of 30,000 plants that will enter recombinant protein production greenhouses. Subsequent batches also consisting of 30000 plants will enter the production greenhouses on an approximately weekly schedule.

The operational basis of the production greenhouse is 100 kg/year of transgenic protein downstream of purification process per year, processed in 50 batches, harvested every 7 days with two weeks down time per year. Protein recovery is estimated in a downstream material balance module for individual unit processes in the separation/purification/formulation process train. Cumulative recovery is calculated at approximately 36% of CEA-based transgenic protein production.

Transgenic plants in the production greenhouse are then cultivated to favor vine growth and maximum expression of the Rubisco gene promoter that is operably linked to the recombinant protein gene. Transgenic plants are grown with 10 hours of light per day, with a light intensity 400 umol/s/m² and a temperature of 24° C. The transgenic plants are grown using variable spacing to accommodate maximum use of lighting, starting in 4 inch diameter pots at approximately 9 plants/ft² with sufficient spacing to accommodate 1.4 plant/ft² at harvest maturity. The mustard greens are harvested starting at day 410 for the first batch, 50 days after transfer to the greenhouse. Expression levels at harvest average 3% total soluble protein for all green tissues. The yield of raw recombinant protein is approximately 280 kg per total progeny (244 kg/acre/yr) micropropagated from the single or multiple plant(s) selected at day 150. Assuming approximately 65% losses associated with harvest and downstream purification of recombinant product, the total manufacturing facility output is 100 kg/yr using approximately 50000 ft² (1.15 acre) of greenhouse floor space.

### Example 7

Selection of Transgenic Plants for CEA Based on In Vitro Testing of Heterologous Protein Stability in Plant Extracts

The stability of human coagulation Factor VIII in plant extracts of Solanum tuberosum L. cv. FL1607 was determined for different leaf positions along the main stem. Leaves were taken from 60-day-old S. tuberosum L. cv. FL1607 plants grown in 6 in soil pots under a 14 hour/day photoperiod. For each leaf position, Coatest activity of “spiked” human coagulation Factor VIII was determined at 0 and 2 hours incubation in plant protein extract. The Coatest assay involved the determination of activation of added coagulation Factor X in the presence of added coagulation Factor IXa and in situ coagulation Factor VIII and provides direct evidence of coagulation Factor VIII concentration (Helena Laboratories, Beaumont, Tex.). The control consisted of a Factor VIII protein standard that did not contain S. tuberosum L. cv. FL1607 plant extract. A comparison was made to the stability of human coagulation Factor VIII in leaf extracts from 60 day-old Nicotiana tabacum L. cv. Xanthi and Medicago sativa L grown in 6-inch soil pots under a 14 hour/day photoperiod.

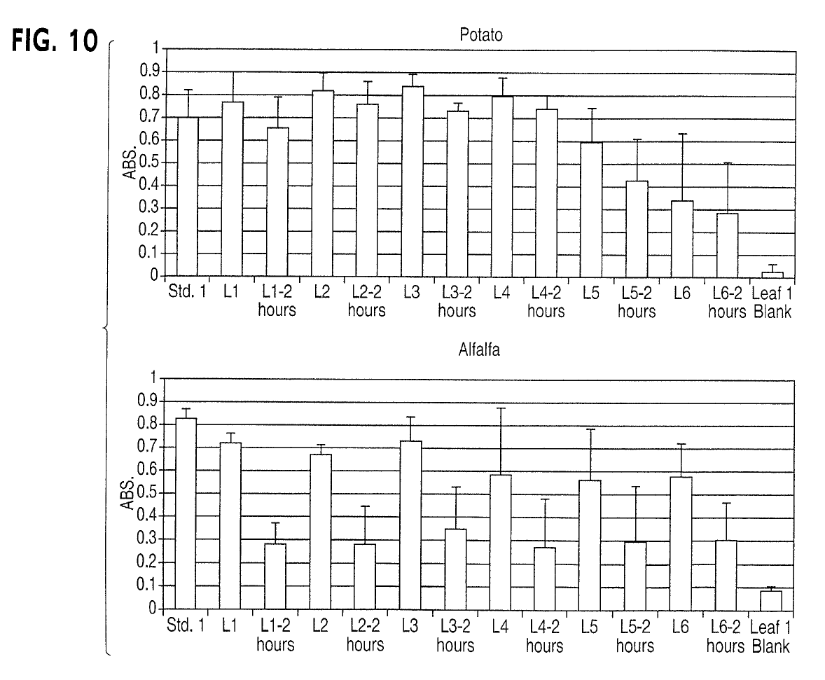

The results of the S. tuberosum, N. tabacum, and M. sativa assays are shown in FIGS. 10A-C, respectively. Human coagulation Factor VIII was most stable in S. tuberosum var. FL1607 leaf extracts with exception to those leaves taken from the very bottom of the S. tuberosum stem (positions 6 and 7). Data for M. sativa, suggest at least moderate proteolysis throughout the tested plants, as Factor VIII activity dropped by at least 50% over the two-hour plant extract incubation period. The strongest proteolytic response was observed for a single test conducted with N. tabacum. In this study, Factor VIII activity at 0 hours was much less than that of a protein buffer standard, suggesting that significant Factor VIII proteolysis occurred within the 5 minute incubation required for activity testing. Further, after 2 hours of incubation in N. tabacum extract, remaining Factor VIII activity was at approximately 20% or less of the original “spiked” amount.

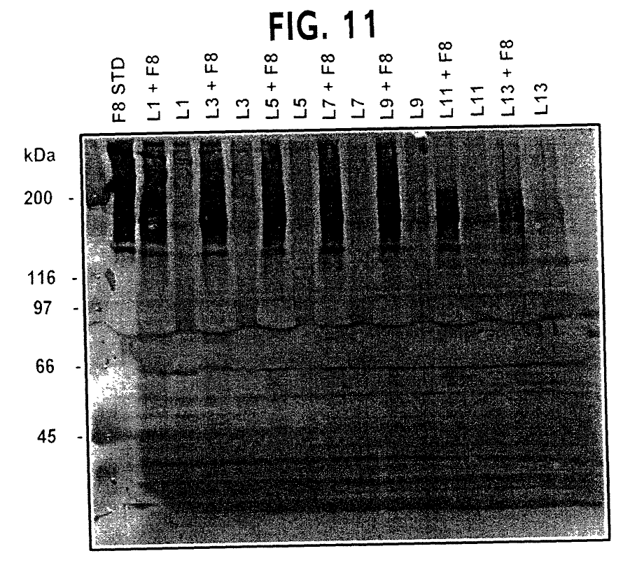

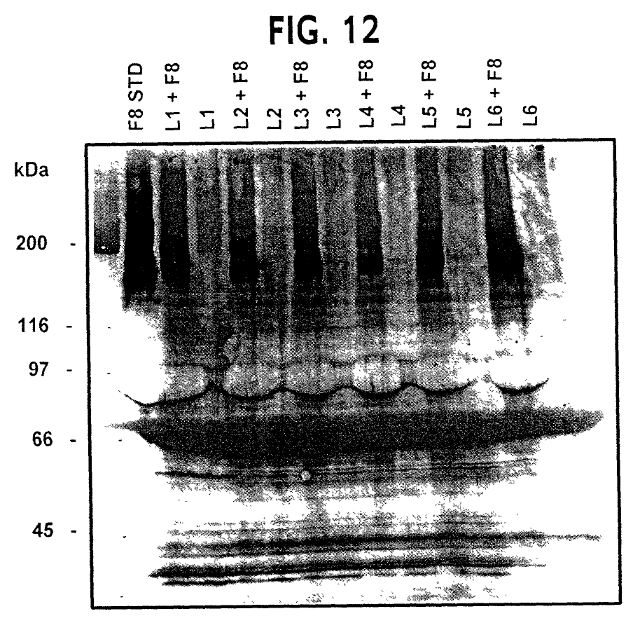

Western blot immunoassays were completed on extracts resulting from tests completed on both S. tuberosum (FIG. 11) and M. sativa (FIG. 12). Protein bands on SDS-PAGE were probed using sheep anti-human coagulation Factor VIII polyclonal antibody. Despite the loss in intensity seen in lanes from 2-hour plant extract treatment, Factor VIII bands (putatively corresponding to light- and heavy-chains, at approximately 150 and 210 kDa, respectively) persist between 0 and 2 hours for potato leaf samples (119). The only exceptions appear in leaf 11 and 13, where the 210 kDa band disappears completely at 2-hour treatment durations and fades significantly even at 0 hours of treatment. It should be noted the proteolysis as compared to standard lanes may occur presumably at 0-hour duration treatment due to the 5 minute sample incubation required to complete the Coatest assay

In contrast to Western blot analysis for S. tuberosum shown in FIG. 11, M. sativa showed complete disappearance of the heavy chain band (at 210 kDa) after 2-hour treatment in all leaf positions except leaf 1. In addition, band intensity at 0-hour treatment is significantly diminished as compared to results for S. tuberosum in FIG. 11, suggesting more robust proteolysis in alfalfa leaf extracts.

What is claimed is:

1. A plant system for producing a heterologous protein under defined, controlled environmental conditions, the plant system comprising a plant (a) transformed with an expression vector comprising a gene coding for the heterologous protein operably linked to a promoter that is selected for optimal expression under the defined environmental conditions of CEA; (b) that produces a large amount of plant biomass under the defined environmental conditions, and (c) that produces tissue and tissue extract wherein the heterologous protein is stable.

2. The plant system of claim 1 wherein the plant is selected from the group consisting of Solanum, Spinacia and Brassica.

3. The plant system of claim 1, wherein the plant is Solanum, the promoter is light-inducible and the defined environmental conditions of CEA include at least 12 hours of light per day.

4. The plant system of claim 1, wherein the promoter is from the ribulose bis-phosphate carboxylase (Rubisco) small subunit gene.

5. The plant system of claim 1, wherein the promoter is CO₂-inducible and the defined environmental conditions include between about 350 and 2,500 ppm CO₂.

6. The plant system of claim 1, wherein the promoter is heat-inducible and the defined environmental conditions include a temperature between about 28 and 40° C.

7. The plant system of claim 6, wherein the heat-inducible promoter is the promoter from the hsp80 gene.

8. The plant system of claim 1, wherein the promoter is a chemically inducible promoter.

9. The plant system of claim 8, wherein the promoter is from the pathogenesis-related beta 1,3 glucanase gene, lipoxygenase 1 gene or potato proteinase inhibitor I gene.

10. The plant system of claim 1, wherein the promoter is a dark-inducible promoter.

11. The plant system of claim 10, wherein the promoter is from the potato proteinase inhibitor I or aminotransferase gene.

12. The plant system of claim 1, wherein the promoter is a constitutive promoter.

13. The plant system of claim 12, wherein the promoter is from the tobacco rpL34 gene, the agrobacterium nopaline synthase gene or the CaMV 35S gene.

14. The plant system of claim 1, wherein the plant is potato which produces between about 0.2 and 5 kilogram fresh weight vines per plant.

15. The plant system of claim 1, wherein the plant is mustard which produces between about 0.2 and 250 grams dry weight greens per plant.

16. A method of producing heterologous protein in a transformed plant comprising the steps of:
a. transforming a plant with an expression vector comprising a gene coding for the heterologous protein operably linked to a promoter that is selected for optimal expression under defined environmental conditions of CEA;
b. cultivating the plant under the defined environment conditions of CEA; and
c. extracting the heterologous protein.

17. The method of claim 16, wherein the plant is selected from the group consisting of Solanum, Spinacia and Brassica.

18. The method of claim 16, wherein the plant is Solanum, the promoter is light-inducible and the defined environmental conditions include at least 12 hours of light per day.

19. The method of claim 18, wherein the promoter is from the Rubisco small subunit gene.

20. The method of claim 16, wherein the promoter is CO₂-inducible and the defined environmental conditions include between about 350 and 2,500 ppm CO₂.

21. The method of claim 16, wherein the promoter is heat-inducible and the defined environmental conditions include a temperature between about 28 and 40° C.

22. The method of claim 21, wherein the heat-inducible promoter is the promoter from the hsp80 gene.

23. The method in claim 16, wherein the promoter is chemically inducible.

24. The method in claim 23, wherein the chemically inducible promoter is from the pathogenesis-related beta 1,3 glucanase gene, lipoxygenase 1 gene or potato proteinase inhibitor I gene.

25. The method of claim 16, wherein the promoter is a dark-inducible promoter.

26. The method of claim 25, wherein the promoter is from the potato proteinase inhibitor I or aminotransferase gene.

27. The method of claim 16, wherein the promoter is a constitutive promoter.

28. The method of claim 27, wherein the promoter is from the tobacco rpL34 gene, the agrobacterium nopaline synthase gene or the CaMV 35S gene.

29. A method of making a plant system for production of a heterologous protein comprising the steps of:
a. identifying a plant that produces a large amount of plant biomass under controlled environmental conditions, that can be rapidly propagated vegetatively and produces tissues and soluble protein extracts that provide increased stability against proteolysis and other damage to heterologous protein targets;
b. transforming the plant with an expression vector comprising a gene coding for the heterologous protein operably linked to a promoter that is selected for optimal expression under the defined environmental conditions of CEA; and
c. selecting a transformed plant that (i) produces a large amount of the heterologous protein and (ii) the heterologous protein is stable in plant tissues and an extract made from the plant.

30. The method of claim 29, wherein the plant is potato and is selected to produce between about 0.2 and 5 kg fresh weight vines per plant.

31. The method of claim 29, wherein the plant is mustard and is selected to produce between about 0.2 and 250 grams dry weight greens per plant.

32. The method of claim 29, wherein the plant is potato and is selected to produce between about 10 and 1300 kg heterologous protein/acre/year.

33. The method of claim 29, wherein the plant is mustard and is selected to produce between about 8 and 1000 kg heterologous protein/acre/year.

34. The method of claim 29, wherein the plant is Solanum, the promoter is light-inducible and the defined environmental conditions include at least 12 hours of light per day.

35. The method of claim 34, wherein the promoter is from the ribulose bis-phosphate carboxylase (Rubisco) small subunit gene.

36. The method of claim 29, wherein the promoter is CO₂-inducible and the defined environmental conditions include between 350 and 2,500 ppm CO₂.

37. The method of claim 29, wherein the promoter is heat-inducible and the defined environmental conditions include a temperature between about 28 to 40° C.

38. The method of claim 37, wherein the heat-inducible promoter is the promoter from the hsp80 gene.

39. The method of claim 29, wherein the promoter is a chemically inducible promoter.

40. The method of claim 39, wherein the promoter is from the pathogenesis-related beta 1,3 glucanase gene, lipoxygenase 1 gene or potato proteinase inhibitor gene.

41. The method of claim 29, wherein the promoter is a dark-inducible promoter.

42. The method of claim 41, wherein the promoter is from the potato proteinase inhibitor I or aminotransferase gene.

43. The method of claim 29, wherein the promoter is a constitutive promoter.

44. The method of claim 43, wherein the promoter is from the tobacco rpL34 gene, the agrobacterium nopaline synthase gene or the CaMV 35S gene.
